(19) United States
(12) Patent Application Publication
(10) Pub. No.: US 2026/0137512 A1
(43) Pub. Date: May 21, 2026

(54) DOCKING STATION WITH IMPROVED SEALING SKIRT

(71) Applicant: Edwards Lifesciences Corporation, Irvine, CA (US)

(72) Inventors: Michael G. Valdez, Riverside, CA (US); Amirsepehr Azimian, Costa Mesa, CA (US); Omar Fawzi Azanki, Coto de Caza, CA (US); Anne Bernadette Aragon Alcasid, Costa Mesa, CA (US); David Lyle Leibowitz, Capistrano Beach, CA (US); Tiana Tran, Westminster, CA (US)

(21) Appl. No.: 19/451,285

(22) Filed: Jan. 16, 2026

Related U.S. Application Data
(63) Continuation of application No. PCT/US2024/038243, filed on Jul. 16, 2024.
(60) Provisional application No. 63/514,330, filed on Jul. 18, 2023.

(51) Int. Cl. A61F 2/24

(52) U.S. Cl. CPC A61F 2/2418; A61F 2/2436; A61F 2220/0075; A61F 2230/0006; A61F 2250/0037; A61F 2250/0069; A61F 2250/0098

(57) ABSTRACT

An implant may include a frame including outer struts to interface with a patient; and inner struts that form a valve seat within a central cavity of the frame. The implant may also include a sealing skirt overlaying the frame from the valve seat to the outer struts at a distal end of the frame, extending along a span of the outer struts, and then extending back towards the inner struts to enclose an annular region about the valve seat.

### CROSS REFERENCE TO RELATED APPLICATIONS

This is a continuation of PCT Patent Application No. PCT/US2024/038243, filed on Jul. 16, 2024, which claims the benefit of U.S. Provisional Ser. No. 63/514,330 , filed Jul. 18, 2023, each of these applications being incorporated herein in its entirety by this reference.

### FIELD

The present disclosure relates to implantable adaptor systems for engaging and retaining a prosthetic implant such as a prosthetic heart valve in a lumen of the body, such as a blood vessel or valve of the heart.

### BACKGROUND

Prosthetic heart valves may be used to treat cardiac valvular disorders. The native heart valves (the aortic, pulmonary, tricuspid, and mitral valves) function to prevent backward flow or regurgitation, while allowing forward flow. These heart valves may be rendered less effective by congenital, inflammatory, infectious conditions, etc. Such conditions may eventually lead to serious cardiovascular compromise or death. For many years, doctors attempted to treat such disorders with surgical repair or replacement of the valve during open heart surgery.

A transcatheter technique for introducing and implanting a prosthetic heart valve using a catheter in a manner that is less invasive than open heart surgery may reduce complications associated with open heart surgery. In this technique, a prosthetic valve may be mounted in a crimped state on the end portion of a catheter and advanced through a blood vessel of the patient until the valve reaches the implantation site. The valve at the catheter tip may then be expanded to its functional size at the site of the defective native valve, such as by inflating a balloon on which the valve is mounted or, for example, the valve may have a resilient, self-expanding stent or frame that expands the valve to its functional size when it is advanced from a delivery sheath at the distal end of the catheter. Optionally, the valve may have a balloon-expandable frame, self-expanding frame, a mechanically-expandable frame, and/or a frame expandable in multiple or a combination of ways.

Transcatheter heart valves (THVs) may be appropriately sized for placement inside many native cardiac valves or orifices. However, with larger native valves, blood vessels (e.g., an enlarged aorta), grafts, etc., aortic transcatheter valves might be too small to secure into the larger implantation or deployment site. In this case, the transcatheter valve may not be large enough to sufficiently expand inside the native valve or other implantation or deployment site or the implantation/deployment site may not provide a good seat for the THV to be secured in place. As one example, aortic insufficiency may be associated with difficulty securely implanting a THV in the aorta and/or aortic valve. Accordingly, there exists a need for improved systems and methods of securing a THV in a relatively large diameter blood vessel or annulus.

### SUMMARY

Certain embodiments of the disclosure pertain to docking stations, frame adaptors, prestents, and the like for engaging and retaining a prosthetic implant such as a prosthetic heart valve in a lumen of the body, such as a blood vessel or valve of the heart. In a representative embodiment, an implant may include a frame including outer struts to interface with a patient; and inner struts that form a valve seat within a central cavity of the frame. The implant may also include a sealing skirt overlaying the frame from the valve seat to the outer struts at a distal end of the frame, extending along a span of the outer struts, and then extending back towards the inner struts to enclose an annular region about the valve seat.

One or more embodiments of the present disclosure may include an implant that may include a frame including outer struts to interface with a patient; and inner struts that form a valve seat within a central cavity of the frame. The implant may also include a sealing skirt overlaying the frame from the valve seat to the outer struts at a distal end of the frame, where the sealing skirt may include a first side that extends a first distance from the distal end of the frame along the outer struts and a second side that extends a second distance from the distal end of the frame along the outer struts, where the second distance is longer than the first distance.

One or embodiments of the present disclosure may include an implant that may include a frame that may include outer struts; and inner struts that form a valve seat within a central cavity of the frame. The implant may also include a sealing skirt overlaying the frame from the valve seat to the outer struts at a distal end of the frame. The sealing skirt may be coupled to the frame using stitches that are at least partially oriented in a longitudinal direction of the frame.

The foregoing and other objects, features, and advantages of the described technology will become more apparent from the following detailed description, which proceeds with reference to the accompanying figures.

### BRIEF DESCRIPTION OF THE DRAWINGS

FIG. 1A is a cutaway view of the human heart in a diastolic phase.

FIG. 1B is a cutaway view of the human heart in a systolic phase.

FIG. 2A is a cutaway view of the human heart with an embodiment of an example docking station positioned in a blood vessel, the inferior vena cava (IVC).

FIG. 2B is an end view of an example docking station and valve showing the valve in an open configuration such that blood may flow through the valve, e.g., when the heart is in a diastolic phase.

FIG. 2C is an end view of the docking station and valve of FIG. 2B showing the valve in a closed configuration, e.g., when the heart is in a systolic phase.

FIG. 3A is a sectional view of an example embodiment of a docking station with an example transcatheter valve disposed inside the docking station.

FIG. 3B is a top view of the docking station and valve illustrated by FIG. 3A.

FIG. 3C is a perspective view of an example embodiment of a docking station that illustrates an example of a frame portion that may be used in the docking station of FIGS. 3A-3B.

FIG. 3D is a sectional view of the docking station illustrated by FIG. 3A where the transcatheter valve shown is representative of a leaflet type transcatheter valve.

FIGS. 4A and 4B schematically illustrate deployment of a docking station.

FIGS. 4C and 4D schematically illustrate deployment of a valve in the docking station.

FIG. 5 illustrates components of another example implant configured to dock and/or support one or more prosthetic valves and/or valve components.

FIG. 6 illustrates components of an additional example implant configured to dock and/or support one or more prosthetic valves and/or valve components.

FIG. 7A illustrates a side view of components of another example implant configured to dock and/or support one or more prosthetic valves and/or valve components.

FIGS. 7B-7F illustrate an example deployment of the implant illustrated in FIG. 7A.

FIG. 8A illustrates a cut-away front view of an example implant configured to dock and/or support one or more prosthetic valves and/or valve components with a sealing skirt enclosing an annular region.

FIG. 8B illustrates a top-down view of the implant of FIG. 8A.

FIG. 8C illustrates a cut-away front view of the implant of FIG. 8A when deployed within a patient.

FIG. 8D illustrates a front view of the implant of FIG. 8A.

FIG. 9A illustrates a cut-away front view of an example implant configured to dock and/or support one or more prosthetic valves and/or valve components with an asymmetric sealing skirt.

FIG. 9B illustrates a top-down view of the implant of FIG. 9A.

FIG. 9C illustrates a cut-away front view of the implant of FIG. 9A when deployed within a patient.

FIG. 9D illustrates a front view of the implant of FIG. 9A.

FIG. 10 illustrates a cut-away front view of an example implant configured to dock and/or support one or more prosthetic valves and/or valve components with improved stitches for the sealing skirt.

FIG. 11A illustrates a front view of another example implant configured to dock and/or support one or more prosthetic valves and/or valve components with stitches for the sealing skirt.

FIG. 11B illustrates a front view of the implant of FIG. 11A with improved stitches for the sealing skirt.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

It should be understood that the disclosed embodiments may be adapted for delivery and implantation in any of the native annuluses and blood vessels of the heart (e.g., the pulmonary, mitral, and tricuspid annuluses, the inferior and superior vena cava, etc.), and may be used with any of various delivery approaches (e.g., retrograde, antegrade, transseptal, transventricular, transatrial, etc.).

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods may be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, may be used in combination with any other feature described herein.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

In the context of the present application, the terms “lower” and “upper” are used interchangeably with the terms “inflow” and “outflow”, respectively. Thus, for example, typically the lower end of a valve or docking station as depicted in the figures is its inflow end and the upper end of the valve or docking station is its outflow end unless explicitly described otherwise.

As used herein, the term “proximal” refers to a position, direction, or portion of a device that is closer to the user (e.g., clinician) and further away from the implantation site and/or body lumen orifice. As used herein, the term “distal” refers to a position, direction, or portion of a device that is further away from the user and closer to the implantation site and/or body lumen orifice. Thus, for example, proximal motion of a device is motion of the device toward the user, while distal motion of the device is motion of the device away from the user.

The terms “longitudinal” and “axial” refer to an axis extending in the upstream and downstream directions, or in the proximal and distal directions, unless otherwise expressly defined.

Although there are alternatives for various components, features, parameters, operating conditions, etc., set forth herein, that does not mean that those alternatives are necessarily equivalent and/or perform equally well. Nor does it mean that the alternatives are listed in a preferred order unless stated otherwise.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part may become a “lower” part simply by turning the object over. Nevertheless, it is still the same part and the object remains the same. As used herein, “and/or” means “and” or “or”, as well as “and” and “or”.

As used herein, the terms “integrally formed” and “unitary construction” refer to a construction that does not require any sutures, fasteners, or other securing means to attach two portions of the construction together.

### Examples of Disclosed Technology

The present disclosure pertains to valve adapter/docking station/landing zone/prestent technology for implanting a prosthetic heart valve, such as a transcatheter heart valve, in a lumen or valve of the heart where the diameter of the lumen or valve is significantly greater than the functional diameter of the prosthetic valve. In certain examples, the docking station may comprise a radially expandable and collapsible frame formed from a plurality of struts, and including a valve seat within the frame configured to receive an expandable prosthetic valve. In certain embodiments, the valve seat may comprise a plurality of struts coupled to the frame and angled inwardly toward the longitudinal axis of the frame. The valve seat may be configured to engage and retain prosthetic valves of a variety of types and sizes. The outer aspect of the docking station frame may engage the surrounding tissue of the native lumen and form a seal, and the valve seat may engage and retain the prosthetic heart valve within the docking station. In certain embodiments, the frame may comprise a sealing member configured to form a seal between the frame and the surrounding anatomy without substantially interfering with blood flow entering the upstream portions of the frame, such as adjacent the ostia of the hepatic veins when implanted in the inferior vena cava.

In certain embodiments, the struts of the valve seat may form valve seat frame cells of the frame. In certain embodiments, struts and/or cells of the valve seat may comprise free end portions/apices, which may be disposed within the lumen of the docking station frame and define a reduced diameter portion configured to engage and retain a prosthetic heart valve. In certain embodiments, the struts of the valve seat may be coupled to the docking station frame at frame junctions, and the free end portions/apices of the valve seat may be offset from the frame junctions in a downstream direction toward the outflow end of the frame. This reduce or minimize the length of the prosthetic valve that protrudes or extends distally or in the downstream direction from the docking station. In certain embodiments, the struts of the valve seat may be wholly disposed within the docking station frame, or the free end portions/apices of the valve seat may define a downstream-most end of the docking station frame.

In certain embodiments, the docking station frame may comprise a plurality of circumferentially-arranged vertical struts. The vertical struts may reduce or prevent foreshortening of the frame between the collapsed and expanded configuration. This may facilitate more accurate and/or predictable deployment of the docking station from the collapsed delivery configuration. The vertical struts may also facilitate recapture of the docking station frame from a partially deployed state by limiting an angle formed by the flared, partially deployed portion of the frame and the longitudinal axis of the delivery apparatus. The vertical struts may also strengthen the frame and reduce or eliminate infolding or invagination of the frame during recapture.

In certain embodiments, the docking station frame may comprise a plurality of free end portions or apices arranged circumferentially around the frame. In certain embodiments, the free apices may be located between pairs of adjacent vertical struts. In certain embodiments, the free apices may be proximal and/or distal apices of frame cells defined between pairs of longitudinal frame struts. In certain embodiments, the frame cells may be axially spaced apart from each other. The free apices may be configured to engage the surrounding tissue of a body lumen in which the docking station frame is implanted to prevent frame movement/migration/rotation relative to the body lumen.

In some embodiments, docking stations/devices for prosthetic valves or THVs are illustrated as being used within the superior vena cava (SVC), inferior vena cava (IVC), or both the SVC and the IVC, although the docking stations/devices (e.g., docking station/device 10, other docking stations/devices described herein, modified versions of the docking stations, etc.) may be used in other areas of the anatomy, heart, or vasculature, such as the tricuspid valve, the pulmonary valve, the pulmonary artery, the aortic valve, the aorta, the mitral valve, or other locations. The docking stations/devices described herein may be configured to compensate for the deployed transcatheter valve or THV being smaller and/or having a different geometrical shape than the space (e.g., anatomy/heart/vasculature/etc.) in which it is to be placed. For example, the native anatomy (e.g., the IVC) may be oval, egg shaped, or another shape, while the prosthetic valve or THV may be cylindrical.

Various embodiments of docking stations/devices and examples of prosthetic valves or transcatheter valves are disclosed herein, and any combination of these options may be made unless specifically excluded. For example, any of the docking stations/devices disclosed, may be used with any type of valve, and/or any delivery system, even if a specific combination is not explicitly described. Likewise, the different constructions and features of docking stations/devices and valves may be mixed and matched, such as by combining any docking station type/feature, valve type/feature, covering/sealing element, etc., even if not explicitly disclosed. In short, individual components of the disclosed systems may be combined unless mutually exclusive or physically impossible.

For the sake of uniformity, in the present disclosure the docking stations are typically depicted such that the right atrium end (e.g., the outflow end) is up, while the ventricular end or IVC end (e.g., the inflow end) is down unless otherwise indicated.

### First Representative Embodiment

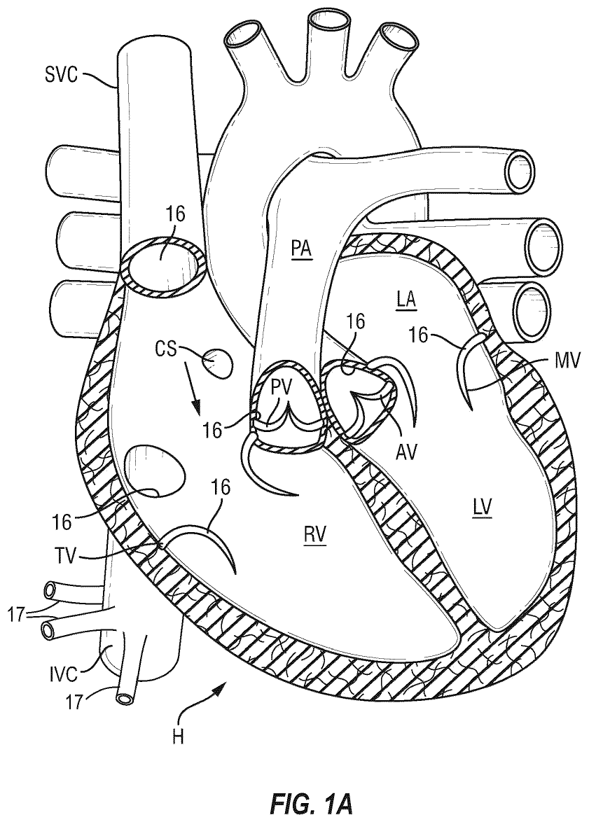

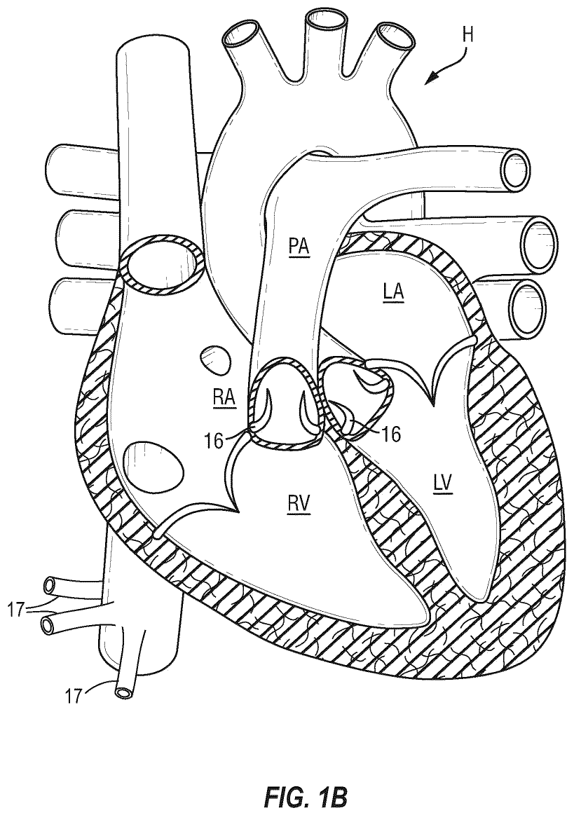

FIGS. 1A and 1B are cutaway views of the human heart (H) in diastolic and systolic phases, respectively. The right ventricle (RV) and left ventricle (LV) are separated from the right atrium (RA) and left atrium (LA), respectively, by the tricuspid valve (TV) and the mitral valve (MV); i.e., the atrioventricular valves. Additionally, the aortic valve (AV) separates the left ventricle (LV) from the ascending aorta (not identified) and the pulmonary valve (PV) separates the right ventricle from the pulmonary artery (PA). Each of these valves has flexible leaflets extending inward across the respective orifices that come together or “coapt” in the flowstream to form one-way, fluid-occluding surfaces. The docking stations and valves of the present application are described, for illustration, primarily with respect to the inferior vena cava (IVC), superior vena cava (SVC), and aorta/aortic valve. A defective aortic valve, for example, may be a stenotic aortic valve and/or suffer from insufficiency and/or regurgitation. The blood vessels, such as the aorta, IVC, SVC, pulmonary artery, may be healthy or may be dilated, distorted, enlarged, have an aneurysm, or be otherwise impaired. Anatomical structures of the right atrium RA, right ventricle RV, left atrium LA, and left ventricle LV will be explained in greater detail. The devices described herein may be used in various areas whether explicitly described herein or not, e.g., in the IVC and/or SVC, in the aorta (e.g., an enlarged aorta) as treatment for a defective aortic valve, in other areas of the heart or vasculature, in grafts, etc.

The right atrium RA receives deoxygenated blood from the venous system through the superior vena cava SVC and the inferior vena cava IVC, the former entering the right atrium from above, and the latter from below. The hepatic veins 17 carry blood from the liver to the inferior vena cava IVC. The coronary sinus (CS) is a collection of veins joined together to form a large vessel that collects deoxygenated blood from the heart muscle (myocardium), and delivers it to the right atrium RA. During the diastolic phase, or diastole, seen in FIG. 1A, the deoxygenated blood from the IVC, SVC, and CS that has collected in the right atrium RA passes through the tricuspid valve TV and into the RV as the right ventricle RV expands. In the systolic phase, or systole, seen in FIG. 1B, the right ventricle RV contracts to force the deoxygenated blood collected in the RV through the pulmonary valve PV and pulmonary artery into the lungs.

The devices described herein may be used to supplement the function of a defective tricuspid valve and/or to prevent too much pressure from building up in the RA. During systole, the leaflets of a normally functioning tricuspid valve TV close to prevent the venous blood from regurgitating back into the right atrium RA. When the tricuspid valve does not operate normally, blood may backflow or regurgitate into the right atrium RA, the inferior vena cava IVC, the superior vena cava SVC, and/or other vessels in the systolic phase. Blood regurgitating backward into the right atrium increases the volume of blood in the atrium and the blood vessels that direct blood to the heart. This may cause the right atrium to enlarge and cause blood pressure to increase in the right atrium and blood vessels, which may cause damage to and/or swelling of the liver, kidneys, legs, other organs, etc. A transcatheter valve (THV) implanted in the inferior vena cave IVC and/or the superior vena cava SVC may prevent or inhibit blood from backflowing into the inferior vena cave IVC and/or the superior vena cava SVC during the systolic phase.

The length L, diameter D, and curvature or contour may vary greatly between the superior vena cava SVC and inferior vena cava IVC of different patients. The relative orientation and location of the IVC and/or SVC may also vary between patients. Further, the size or diameter D may vary significantly along the length L of an individual IVC and/or SVC. Also, the anatomy of the IVC and/or SVC is soft, flexible, and dynamic as compared to other cardiac vessels, such as the aorta. This softer, more flexible, and/or more dynamic (moving and/or shape changing) characteristic of the IVC and SVC make it more difficult for a transcatheter valve frame or a docking station that supports a transcatheter valve to anchor in the IVC and/or the SVC than in the aorta. Further, other regions or other vasculature in other areas of the body and across patients where docking stations could be used may also vary significantly in shape and size.

The left atrium LA receives oxygenated blood from the left and right pulmonary veins, which then travels through the mitral valve to the left ventricle. During the diastolic phase, or diastole, seen in FIG. 1A, the oxygen rich blood that collects in the left atrium LA passes through the mitral valve MV and into the left ventricle LV as the left ventricle LV expands. In the systolic phase, or systole, seen in FIG. 1B, the left ventricle LV contracts to force the oxygen rich blood through the aortic valve AV and aorta into the body through the circulatory system. In certain embodiments, the devices described herein may be used to supplement or replace the function of a defective aortic valve. For example, the devices herein may be particularly effective for treating aortic insufficiency. During diastole, the leaflets of a normally functioning aortic valve AV close to prevent the oxygen rich blood from regurgitating back into the left ventricle LV. When the aortic valve does not operate normally, blood backflows or regurgitates into the left ventricle LV. A THV implanted in the aortic valve helps prevent or inhibit blood from backflowing into the left ventricle LV during the diastole phase. The length L, diameter, D, and curvature or contour of the aortic root may vary greatly between different patients, especially if the aorta is dilated, distorted, or enlarged. Further, the size or diameter D may vary significantly along the length L of an individual aorta.

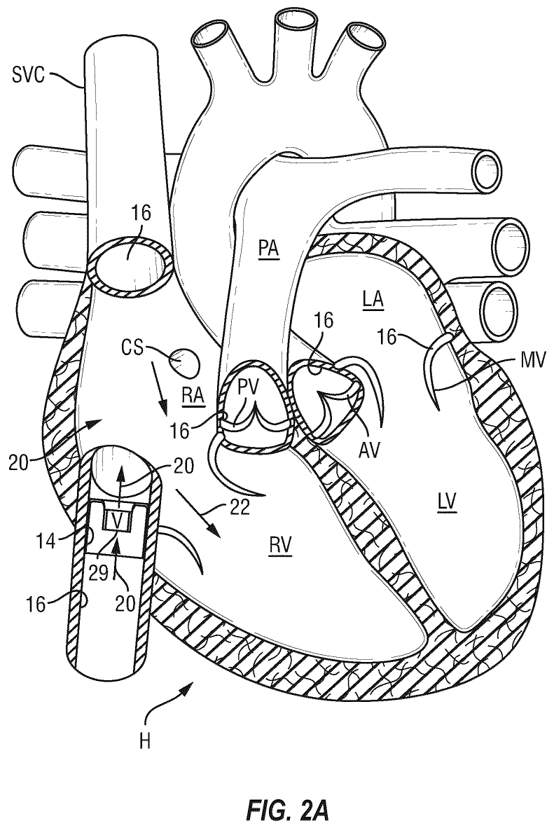

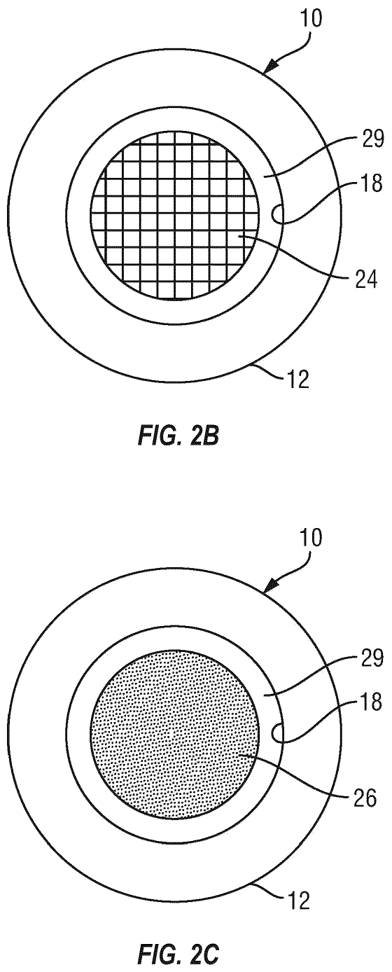

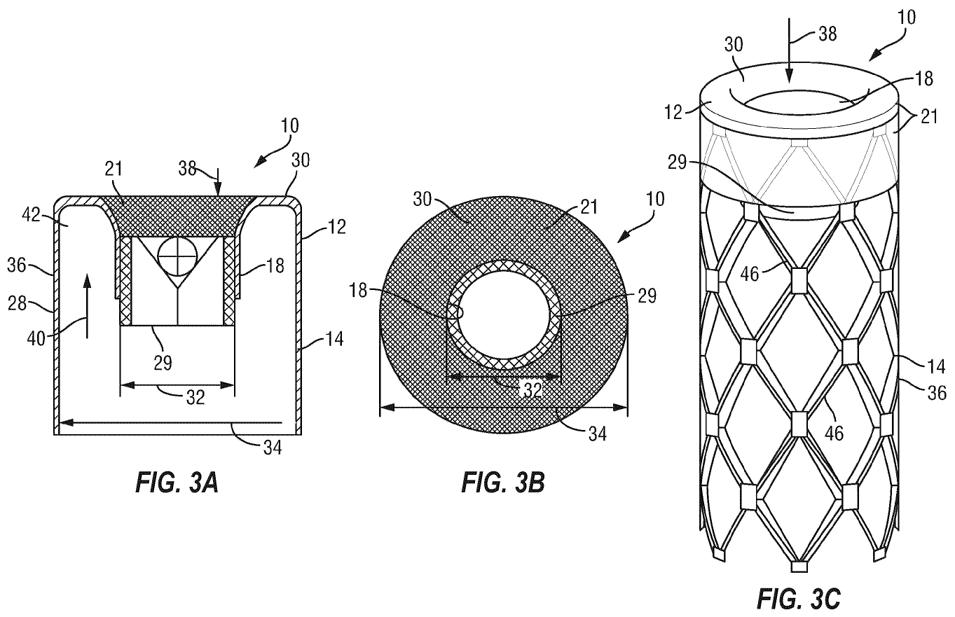

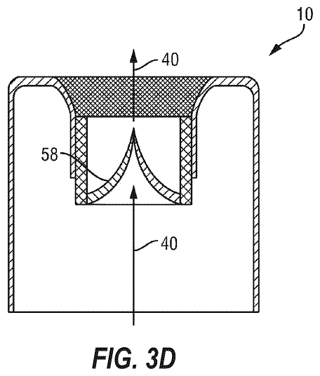

Referring to FIGS. 2A, 3A, 3B, and 3C, in some embodiments an expandable docking station/device/valve adapter/landing zone/prestent 10 includes one or more sealing portions 12, a valve seat 18, and one or more retaining portions 14. The sealing portion(s) 12 provide a seal between the docking station 10 and an interior surface 16 (see FIG. 2A) of the circulatory system. The valve seat 18 provides a supporting surface for implanting or deploying a valve 29 in the docking station 10 after the docking station 10 is implanted in the circulatory system. Optionally, the docking station 10 and the valve 29 may be integrally formed, for example, in one embodiment, the valve seat 18 may be omitted. When integrally formed, the docking station 10 and the valve 29 may be deployed as a single device, rather than first deploying the docking station 10 and then deploying the valve 29 into the docking station. Any of the docking stations and/or valve seats 18 described herein may be provided or formed with an integrated valve 29.

The retaining portion 14 helps retain the docking station 10 and the valve 29 at the implantation position or deployment site in the circulatory system. The retaining portion 14 may take a wide variety of different forms. In some embodiments, the retaining portion 14 includes friction enhancing features that reduce or eliminate migration of the docking station 10. The friction enhancing features may take a wide variety of different forms. For example, the friction enhancing features may comprise barbs, spikes, texturing, adhesive, and/or a cloth or polymer cover with high friction properties on the retaining portions 14. Such friction enhancing features may also be used on any of the various docking stations or retaining portions described herein.

Expandable docking station 10 and valve 29 as described in the various embodiments herein are also representative of a variety of docking stations and/or valves described herein or that might be known or developed, e.g., a variety of different types of valves could be substituted for and/or used as valve 29 in the various docking stations.

FIGS. 2A, 2B, and 2C illustrate a representative example of the operation of the docking stations 10 and valves 29 disclosed herein. In the example of FIGS. 2A, 2B, and 2C, the docking station 10 and valve 29 are deployed in the inferior vena cava IVC. However, the docking station 10 and valve 29 may be deployed in any interior surface within the heart or a lumen of the body. For example, the various docking stations and valves described herein may be deployed in the superior vena cava SVC, the tricuspid valve TV, the pulmonary valve PV, pulmonary artery, the mitral valve MV, the aortic valve AV, aorta, or other vasculature/lumens in the body.

FIGS. 2A and 2B illustrate the valve 29, docking station 10 and heart H, when implanted in the IVC and the heart H is in the diastolic phase. When the heart is in the diastolic phase, the valve 29 opens. Blood flows from the inferior vena cava IVC and the superior vena cava SVC, into the right atrium RA. The blood that flows from the inferior vena cava IVC flows through the docking station 10 and valve 29 as indicated by arrows 20. Also, while in the diastolic phase, blood in the right atrium flows through the tricuspid valve TV, and into the right ventricle RV and valve as indicated by arrows 22. FIG. 2B illustrates space 24 that represents the valve 29 being open when the heart is in the diastolic phase. A variety of types of valves may be used that may open and close in a variety of ways (e.g., including valves with leaflets of tissue that open then coapt to close), so the drawings are meant to be representative of a variety of valves that may operate in different ways. FIG. 2B does not show the interface between the docking station 10 and the inferior vena cava to simplify the drawing. The cross-hatching in FIG. 2B represents blood flow through the valve 29. In some embodiments, blood is prevented or inhibited from flowing between the inferior vena cava IVC and the docking station 10 by the sealing portion 12 and blood is prevented or inhibited from flowing between the docking station 10 and the valve by implanting or seating the valve in the seat 18 of the docking station 10. In this example, blood only substantially flows or is only able to flow through the valve 29 when the valve is open (e.g., in certain embodiments, only when the heart is in the diastolic phase).

FIG. 2C illustrates the valve 29 and docking station 10, when the valve 29 is closed (e.g., when implanted in the IVC and the heart H is in the systolic phase). When implanted in the IVC and the heart is in the systolic phase, the valve 29 closes. Blood is prevented from flowing from the right atrium RA into the inferior vena cava IVC by the valve 29 being closed. As such, the closed valve 29 prevents any blood that regurgitates through the tricuspid valve TV during the systolic phase from being forced into the inferior vena cava IVC. The solid area 26 in FIG. 2C represents the valve 29 being closed (e.g., in certain embodiments, when the heart is in the systolic phase). FIG. 2C is meant to be representative of a variety of valves, even though those valves may close in different ways.

In some embodiments, the docking station 10 acts as an isolator that prevents or substantially prevents radial outward forces of the valve 29 from being transferred to the inner surface 16 of the circulatory system. In one embodiment, the docking station 10 includes a valve seat 18 that resists expansion, e.g., is not expanded radially outwardly (e.g., the diameter of the valve seat does not increase) or is not substantially expanded radially outward (e.g., the diameter of the valve seat increases by less than 4 mm) by the radially outward force of the transcatheter valve or valve 29. The valve seat may be configured such that expansion of a THV/valve 29 increases the diameter of the valve seat only to a diameter less than an outer diameter of the docking station 10 when the docking station is implanted. Retaining portions 14 and sealing portions 12 may be configured to impart only relatively small radially outward forces on the inner surface 16 of the circulatory system (as compared to the radially outward force applied to the valve seat 18 by the valve 29). Having a valve seat 18 that is stiffer or less radially expansive than the outer portions of the docking station (e.g., retaining portions 14 and sealing portions 12), as in the various docking stations described herein, provides many benefits, including allowing a THV/valve 29 to be implanted in vasculature or tissue of varying strengths, sizes, and/or shapes. The outer portions of the docking station may better conform to the anatomy (e.g., vasculature, tissue, heart, etc.) without putting too much pressure on the anatomy, while the THV/valve 29 may be firmly and securely implanted in the valve seat 18 with forces that will prevent or mitigate the risk of migration or slipping.

The docking station 10 may include any combination of one or more than one different types of valve seats 18, retaining portions 14, and/or sealing portions 12. For example, the valve seat 18 may be a separate component that is attached to the frame 28 of the docking station 10, while the sealing portion is integrally formed with the frame 28 of the docking station. Also, the valve seat 18 may be a separate component that is attached to the frame 28 of the docking station 10, while the sealing portion 12 is a separate component that is also attached to the frame 28 of the docking station. Optionally, the valve seat 18 may be integrally formed with the frame 28 of the docking station 10, while the sealing portion is integrally formed with the frame 28 of the docking station. Further, the valve seat 18 may be integrally formed with the frame 28 of the docking station 10, while the sealing portion is a separate component that is attached to the frame 28 of the docking station 10.

The sealing portion 12, the valve seat 18, and one or more retaining portions 14 of the various docking stations herein may take a variety of different forms and characteristics. In FIGS. 3A-3C, an expandable frame 28 provides the shape of the sealing portion 12, the valve seat 18, and the retaining portion 14. The expandable frame 28 may take a wide variety of different forms. The illustrated expandable frame 28 in FIGS. 3A-3C has an end 30 having an inside diameter 32 and an outside diameter 34. An annular or cylindrical outer portion or wall 36 extends downward from the outside diameter 34 of the end 30. An annular or cylindrical valve seat 18 or wall extends downward from the inside diameter 32 of the end 30. In the illustrated example, the expandable frame 28 is an expandable lattice. The expandable lattice may be made in a variety of ways, e.g., with individual wires connected to form the lattice, braiding, cut from a sheet and then rolled or otherwise formed into the shape of the expandable frame, molded, cut from a cylindrical tube (e.g., cut from a nitinol tube), other ways, or a combination of these.

The frame 28 may be made from a highly flexible metal, metal alloy, or polymer. Examples of metals and metal alloys that may be used include, but are not limited to, nitinol and other shape memory alloys, elgiloy, and stainless steel, but other metals and highly resilient or compliant non-metal materials may be used to make the frame 28. These materials may allow the frame to be compressed to a small size, and then when the compression force is released, the frame will self-expand back to its pre-compressed diameter and/or the frame may be expanded by inflation of a device positioned inside the frame. The frame 28 may also be made of other materials and be expandable and collapsible in different ways, e.g., mechanically-expandable, balloon-expandable, self-expandable, or a combination of these.

The sealing portions may take a wide variety of different forms. In the example of FIGS. 3A-3C, a covering/material 21 is attached to a portion of the frame 28 to form the sealing portion 12. However, the sealing portion 12 may be formed in a wide variety of other ways. The covering/material 21 may be a fabric material, polymer material, or other material. The sealing portion 12 may take any form that prevents or inhibits the flow of blood from flowing around the outside surface of the valve 29 and through the docking station. In the example of FIGS. 3A, 3B, and 3C, the sealing portion 12 comprises a covering/material 21 (e.g., a fabric or other covering material that may be the same as or similar to other coverings/materials described herein) that extends up to the valve seat 18. The covering/material 21 may be shaped and positioned in a variety of ways, e.g., the covering/material may be configured to partially cover the valve seat 18, entirely cover the valve seat 18, or not cover the valve seat 18 when the frame 28 is expanded. The covering/material 21 (e.g., fabric or other covering material) that forms the sealing portion 12 may also extend radially outward, covering the end 30 of the frame 28, and may optionally extend (e.g., longitudinally, downward, etc.) to cover at least a portion of the annular outer portion or wall 36. The sealing portion 12 provides a seal between the docking station 10 and an interior surface 16 (see FIG. 2) of the circulatory system. That is, the sealing portion 12 and the closed valve 29 prevent or inhibit blood from flowing in the direction indicated by arrow 38. In the example of FIGS. 3A and 3B, blood is not inhibited from flowing in the direction indicated by arrow 40 into the area 42 between the valve seat 18 and the annular outer portion or wall 36.

The valve seat may take a wide variety of different forms. The valve seat 18 is a portion of the frame 28 in the example of FIGS. 3A-3C. However, the valve seat 18 may be formed separately from the frame 28. The valve seat 18 may take any form that provides a supporting surface for implanting or deploying a valve 29 in the docking station 10 after the docking station 10 is implanted in the circulatory system. The valve seat may optionally be reinforced with a reinforcing material (e.g., a suture, wire, band, collar, etc. that may circumscribe the valve seat or a portion of the valve seat). The valve 29 is schematically illustrated in FIG. 3A to indicate that the valve 29 may take a wide variety of different forms. FIG. 3D illustrates the more specific example where the valve 29 is a leaflet type THV, such as the SAPIEN® 3 valve available from Edwards Lifesciences Corporation, including a plurality of leaflets 58. In some embodiments, a valve 29 is integrated with or replaces the valve seat 18, such that docking station 10 is configured as a transcatheter valve that is delivered as a single unit in the same step (as opposed to first implanting a docking stations and subsequently implanting a separate valve/THV in the docking station). Optionally, any of the docking stations described herein may be formed as a valve or THV, e.g., with valve tissue or other valve material integrated into the docking station.

The retaining portions 14 may take a wide variety of different forms. For example, the retaining portion(s) 14 may be any structure that sets the position of the docking station 10 in the circulatory system. For example, the retaining portion(s) 14 may press against or into the inside surface 16 or contour/extend around anatomical structures of the circulatory system to set and maintain the position of the docking station 10. The retaining portion(s) 14 may be part of or define a portion of the body and/or sealing portion of the docking station 10 or the retaining portion(s) 14 may be a separate component that is attached to the body of the docking station. The docking station 10 may include a single retaining portion 14 or two, or more than two retaining portions. The retaining portion(s) 14 may include friction enhancing features as discussed above.

In the example of FIGS. 3A-3C, the retaining portion 14 comprises the annular outer portion or wall 36 of the frame 28. A shape set (e.g., a programmed shape of a shape memory material) of annular outer portion or wall 36 may bias the annular outer portion or wall 36 radially outward and into contact with/against the interior surface 16 of the circulatory system to retain the docking station 10 and the valve 29 at the implantation position. In the illustrated embodiment, the retaining portion 14 is elongated to allow a relatively small force to be applied to a large area of the interior surface 16, while the valve 29 may apply a relatively large force to the valve seat 18. For example, the length of the retaining portion 14 may be twice, three times, four times, five times, or greater than five times the outside diameter of the transcatheter valve. Applying a small radially outward force over a larger area may be sufficient to securely hold the docking station in place, and this design/configuration may allow the docking station to conform to the unique shape/size of the anatomy and avoid/reduce the likelihood of damaging relatively weaker native tissue. Thereby the valve 29 may be securely held in a variety of locations and anatomies (e.g., the docking station of FIGS. 3A-D is usable in the IVC, SVC, aorta, etc.).

In certain examples, the retaining portion 14 may comprise the annular outer portion or wall 36 of the frame 28. A shape set (e.g., a programmed shape of a shape memory material) of annular outer portion or wall 36 may bias the annular outer portion or wall 36 radially outward and into contact with/against the interior surface 16 of the aorta to retain the docking station 10 and the valve 29 at the implantation position. In certain examples, the shape set may also be selected to substantially match the shape of a portion of the aorta. The retaining portion 14 may be elongated to allow a relatively small force to be applied to a large area of the interior surface 16, while the valve 29 may apply a relatively large force to the valve seat 18, as discussed above.

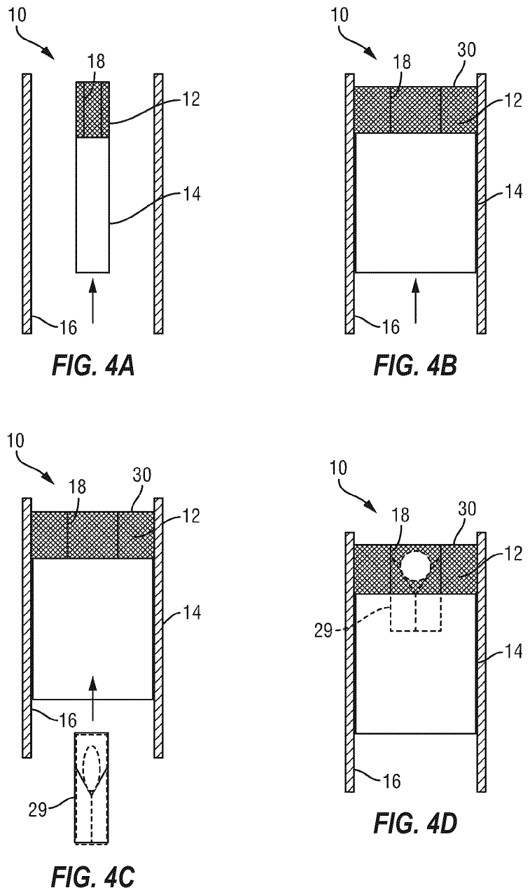

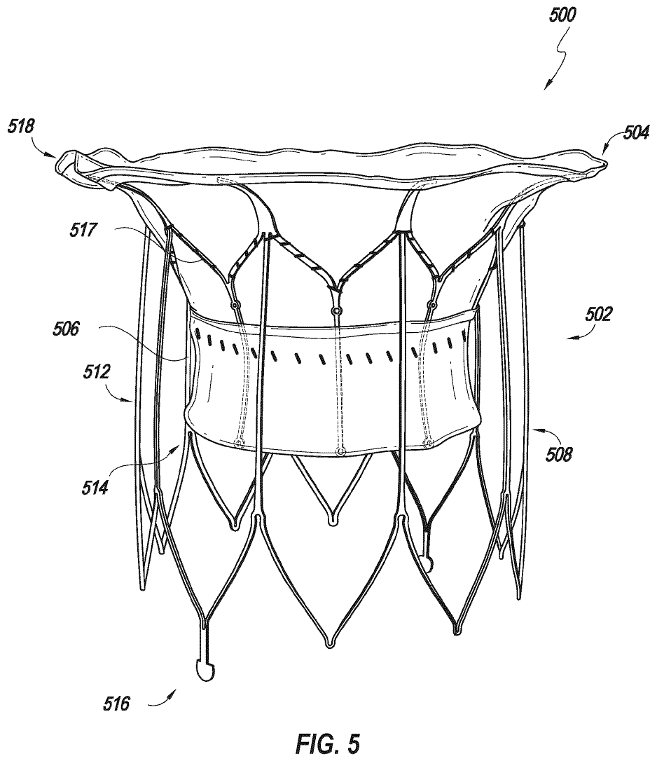

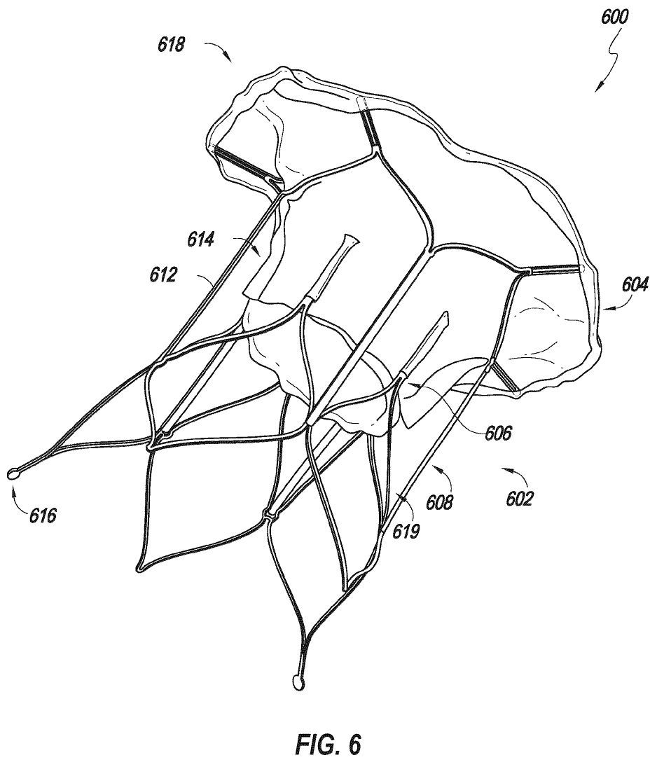

FIGS. 4A-4D schematically illustrate an example deployment of the docking station 10 and valve 29 in the circulatory system. Referring to FIG. 4A, the docking station 10 is in a compressed form/configuration and is introduced to a deployment site in the circulatory system. For example, the docking station 10, may be positioned at a deployment site in a SVC, IVC, aorta, or other location. Referring to FIG. 4B, the docking station 10 is expanded in the circulatory system such that the sealing portion(s) 12 and the retaining portion(s) 14 engage the inside surface 16 of a portion of the circulatory system. The docking station may be self-expanding, and may be advanced from a delivery capsule into the expanded state, or plastically expandable such that it may be expanded using a balloon or other expansion device. Referring to FIG. 4C, after the docking station 10 is deployed, the valve 29 is in a compressed form and is introduced into the valve seat 18 of the docking station 10. Referring to FIG. 4D, the valve 29 is expanded in the docking station, such that the valve 29 engages the valve seat 18 and the seat 18 of the docking station supports the valve. The docking station 10 allows the valve 29 to operate within the expansion diameter range for which it is designed. In the examples depicted herein, the docking station 10 is longer than the valve. However, in some embodiments the docking station 10 may be the same length or shorter than the length of the valve 29. Similarly, the valve seat 18 may be longer, shorter, or the same length as the length of the valve 29. Any of the docking station embodiments described herein may be deployed in the manner described above.

### Second Representative Embodiment

FIG. 5 illustrates components of another example implant 500 configured to dock and/or support one or more prosthetic valves and/or valve components in accordance with one or more instances. The implant 500 may comprise a frame 502 and/or a sealing element 504, which may include a skirt, covering, and/or similar device. The frame 502 may be configured to form an inner frame 506 and/or an outer frame 508. The inner frame 506 may form a first diameter that is less than a second diameter of the outer frame 508. The inner frame 506 and outer frame 508 may be extensions of a common device and/or may extend from each other.

The frame 502 may comprise a wire frame formed by a network of struts 512 (e.g., wires, cords, and/or bars) forming one or more cells 514. The frame 502 may mostly comprise empty space and/or cells 514 between the struts 512. For example, the struts 512 may be generally thin and/or may be spaced apart to create relatively large gaps between the struts 512, as shown in FIG. 5. In some examples, the outer frame 508 may comprise a series of longitudinally extending struts 512 aligned in series and/or in parallel around a circumference of the implant 500. Each of the struts 512 may join to one or more adjacent struts 512 at a proximal end 516 of the implant 500 and/or may join with one or more adjacent struts 512 and/or with the inner frame 506 at a distal end 518 of the implant 500.

The inner frame 506 may similarly comprise a network of generally thin, elongate, and/or spaced apart struts 512. In some examples, one or more struts 512 of the inner frame 506 may extend generally in series and/or in parallel longitudinally along a length of the implant 500. The one or more struts 512 of the inner frame 506 may be disposed between struts 512 of the outer frame 508.

In some examples, the outer frame 508 may have a generally cylindrical form and/or may at least partially enclose a complete circumference of the inner frame 506 and/or of a portion of the sealing element 504. The outer frame 508 may comprise a network of struts 512, which may include wires, arms, bars, cords, walls, and/or similar components forming one or more cells 514 and/or openings through the outer frame 508. The one or more cells 514 may be configured to allow blood flow through the outer frame 508. The cells 514 may have any suitable shape and/or size. The outer frame 508 may form generally elongate cells 514 extending approximately an entire length of the outer frame 508 and/or from a proximal end 516 to a distal end 518 of the implant 500. The one or more cells 514 may have triangular forms at end points of the one or more cells 514. However, the one or more struts 512 may form cells 514 having different shapes. For example, the struts 512 may be configured to form generally rectangular and/or diamond-shaped cells 514. While the struts 512 are shown having generally thick structures, the one or more struts 512 may have wire-like and/or generally thin forms. In some examples, the inner frame 506 and/or outer frame 508 may be configured to maintain a uniform structure and/or strut 512 pattern along a length of the frame 502.

The implant 500 may be configured for delivery and/or placement at an SVC and/or IVC of a heart. For example, the implant 500 may be configured for placement at or near an in-flow junction of the SVC and/or IVC to the right atrium of the heart.

At a distal end 518 of the implant 500, the outer frame 508 and inner frame 506 may join together and/or the inner frame 506 may extend away from the outer frame 508 and/or along an inner lumen formed by the outer frame 508. In some examples, the outer frame 508 and inner frame 506 may both have a generally flared and/or conical form at or near the distal end 518 of the implant 500. The inner frame 506 may have a flaring angle that is greater than the outer frame 508 such that the diameter of the inner frame 506 may be less than the diameter of the outer frame 508. The inner frame 506 may be configured to extend along at least a portion of the length of the outer frame 508.

The flared end (e.g., distal end 518) of the outer frame 508 and/or inner frame 506 may be configured to engage an atrium and/or other chamber when implanted. The sealing element 504 may be configured to extend along the inner frame 506 and/or may be configured to wrap around the outer frame 508 and/or extend between the outer frame 508 and the native tissue. The outer frame 508 may be configured to extend into the IVC and/or other blood vessel and/or the sealing element 504 may be configured to extend from the outer frame 508 to an inner surface of the inner frame 506. The sealing element 504 may be configured for engagement with a prosthetic valve and/or other implant. For example, the sealing element 504 may be configured to provide a mounting surface for a prosthetic valve and/or may be configured to securely hold the prosthetic valve. The sealing element 504 may be configured to extend along only a portion of the implant 500 such that the sealing element 504 may not extend across one or more branching vessels of the blood vessel.

In some examples, the implant 500 may comprise one or more outward bulbs configured to extend outwardly from the diameter of the outer frame 508 to facilitate anchoring of the implant 500 within a blood vessel.

The frame 502 may comprise one or more downward-extending arms 517 (e.g., extending towards the proximal end 516) configured to form the inner frame 506. For example, the arms 517 may extend downwardly from the outer frame 508 at or near the distal end 518 of the frame 502. The one or more arms 517 may be configured to extend at an acute angle away from the outer frame into the lumen of the frame 502 and/or may extend generally in parallel with the outer frame 508 at or near distal ends of the one or more arms 517.

FIG. 6 illustrates components of another example implant 600 configured to dock and/or support one or more prosthetic valves and/or valve components in accordance with one or more instances. The implant 600 may comprise a frame 602 and/or a sealing element 604, which may include a skirt, covering, and/or similar device. The frame 602 may be configured to form an inner frame 606 and/or an outer frame 608. The inner frame 606 may form a first diameter that is less than a second diameter of the outer frame 608. The inner frame 606 and outer frame 608 may be extensions of a common device and/or may extend from each other.

The frame 602 may comprise a wire frame formed by a network of struts 612 (e.g., wires, cords, and/or bars) forming one or more cells 614. Such struts 612 may be referred to as vertical struts. The frame 602 may mostly comprise empty space and/or cells 614 between the struts 612. For example, the struts 612 may be generally thin and/or may be spaced apart to create relatively large gaps between the struts 612, as shown in FIG. 6. In some examples, the outer frame 608 may comprise a series of longitudinally extending struts 612 aligned in series and/or in parallel around a circumference of the implant 600. Each of the struts 612 may join to one or more adjacent struts 612 at a proximal end 616 of the implant 600 and/or may join with one or more adjacent struts 612 and/or with the inner frame 606 at a distal end 618 of the implant 600.

The inner frame 606 may similarly comprise a network of generally thin, elongate, and/or spaced apart struts 612. In some examples, one or more struts 612 of the inner frame 606 may extend generally in series and/or in parallel longitudinally along a length of the implant 600. The one or more struts 612 of the inner frame 606 may be disposed between struts 612 of the outer frame 608.

In some examples, the outer frame 608 may have a generally cylindrical form and/or may at least partially enclose a complete circumference of the inner frame 606 and/or of a portion of the sealing element 604. The outer frame 608 may comprise a network of struts 612, which may include wires, arms, bars, cords, walls, and/or similar components forming one or more cells 614 and/or openings through the outer frame 608. The one or more cells 614 may be configured to allow blood flow through the outer frame 608. The cells 614 may have any suitable shape and/or size. The outer frame 608 may form generally elongate cells 614 extending approximately an entire length of the outer frame 608 and/or from a proximal end 616 to a distal end 618 of the implant 600. The one or more cells 614 may have triangular forms at end points of the one or more cells 614. However, the one or more struts 612 may form cells 614 having different shapes. For example, the struts 612 may be configured to form generally rectangular and/or diamond-shaped cells 614. While the struts 612 are shown having generally thick structures, the one or more struts 612 may have wire-like and/or generally thin forms. In some examples, the inner frame 606 and/or outer frame 608 may be configured to maintain a uniform structure and/or strut 612 pattern along a length of the frame 602.

The implant 600 may be configured for delivery and/or placement at an SVC and/or IVC of a heart. For example, the implant 600 may be configured for placement at or near an in-flow junction of the SVC and/or IVC to the right atrium of the heart.

At a distal end 618 of the implant 600, the outer frame 608 and inner frame 606 may join together and/or the inner frame 606 may extend away from the outer frame 608 and/or along an inner lumen formed by the outer frame 608. In some examples, the outer frame 608 and inner frame 606 may both have a generally flared and/or conical form at or near the distal end 618 of the implant 600. The inner frame 606 may have a flaring angle that is greater than the outer frame 608 such that the diameter of the inner frame 606 may be less than the diameter of the outer frame 608. The inner frame 606 may be configured to extend along at least a portion of the length of the outer frame 608.

The flared end (e.g., distal end 618) of the outer frame 608 and/or inner frame 606 may be configured to engage an atrium and/or other chamber when implanted. The sealing element 604 may be configured to extend along the inner frame 606 and/or may be configured to wrap around the outer frame 608 and/or extend between the outer frame 608 and the native tissue. The outer frame 608 may be configured to extend into the IVC and/or other blood vessel and/or the sealing element 604 may be configured to extend from the outer frame 608 to an inner surface of the inner frame 606. The sealing element 604 may be configured for engagement with a prosthetic valve and/or other implant. For example, the sealing element 604 may be configured to provide a mounting surface for a prosthetic valve and/or may be configured to securely hold the prosthetic valve. The sealing element 604 may be configured to extend along only a portion of the implant 600 such that the sealing element 604 may not extend across one or more branching vessels of the blood vessel.

In some examples, the implant 600 may comprise one or more outward bulbs configured to extend outwardly from the diameter of the outer frame 608 to facilitate anchoring of the implant 600 within a blood vessel.

The frame 602 may comprise one or more upward-extending arms 619 (e.g., extending towards the distal end 618) configured to form the inner frame 606. For example, the arms 619 may extend downwardly from the outer frame 608 at or near the distal end 618 of the frame 602. The one or more arms 619 may be configured to extend at an acute angle away from the outer frame into the lumen of the frame 602 and/or may extend generally in parallel with the outer frame 608 at or near distal ends of the one or more arms 619.

The arms 619 of the implant 600 may extend upwardly from the proximal end 616 to the distal end 618. For example, the one or more arms 619 may extend at an approximately 45-degree angle from the outer frame 608 at or near the distal end 618 of the implant 600 and/or may extend upwardly generally in parallel with the outer frame 608 along a midsection of the implant 600.

### Third Representative Embodiment

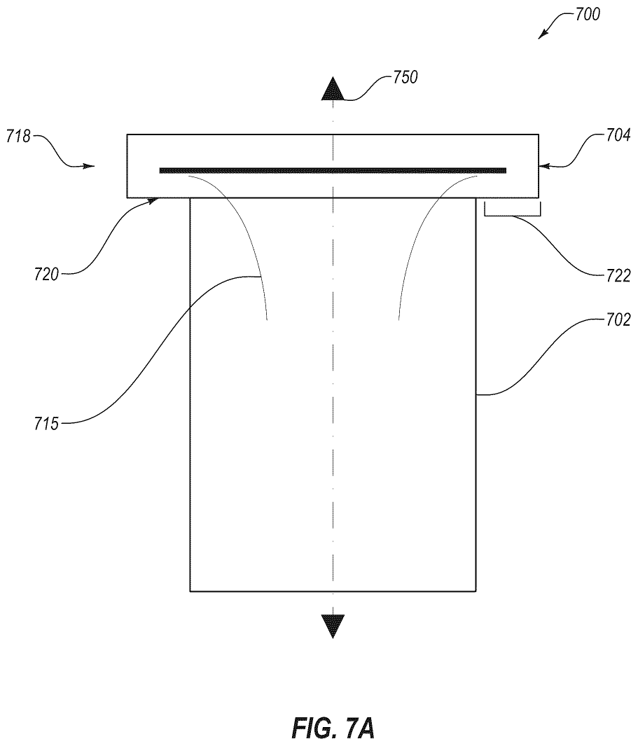

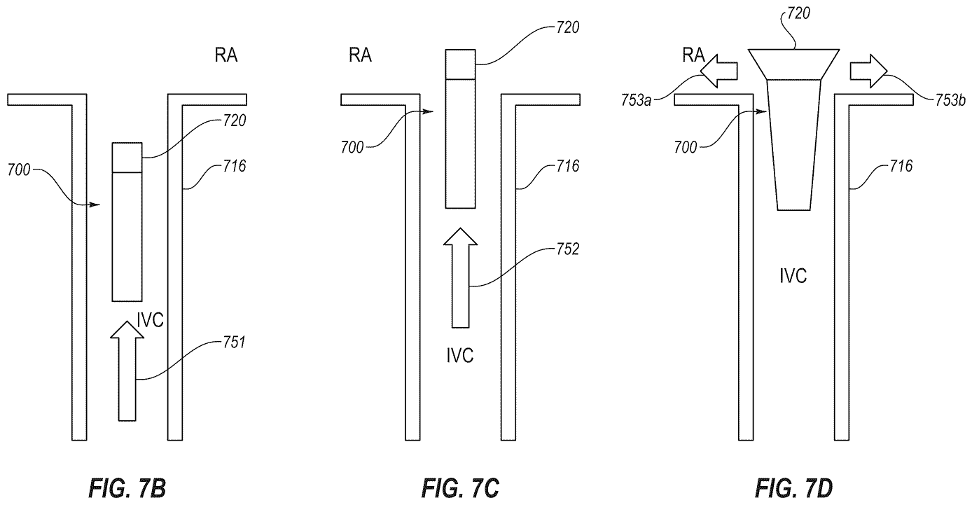

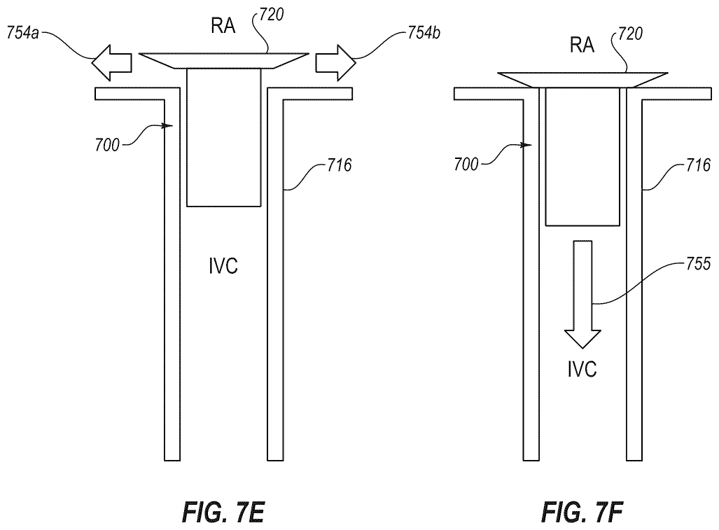

FIG. 7A illustrates a side view of components of another example implant 700 configured to dock and/or support one or more prosthetic valves and/or valve components in accordance with one or more embodiments of the present disclosure. The implant 700 may include a frame 702 and/or a sealing element 704, which may include a skirt, covering, and/or similar device. The frame 702 may be configured to form a valve seat 715. The valve seat 715 may be disposed within a central cavity created by the frame 702. At a distal end 718 of the implant 700, the implant may include a flange 720 to facilitate the sealing element 704 sealing against the anatomy of a patient. For convenience, a central axis 750 of the implant 700 is also illustrated in FIG. 7A.

The flange 720 may provide a projection that extends beyond the remainder of the frame 702. For example, as illustrated in FIG. 7, the flange 720 may produce a lip 722 that is shaped or configured to abut anatomy of the patient. For example, rather than being disposed wholly within the IVC (such as the embodiment illustrated in FIG. 2A), the distal end 718 of the implant 700 may extend into the right atrium with the flange 720 resting against the sidewall of the right atrium proximate where the IVC enters the right atrium (RA). By providing the lip 722, a better seal may be created by the sealing element 704 as compared to embodiments that are radially pressed against the IVC, such as that illustrated in FIG. 2A.

In some embodiments, the shape or profile of the frame 702 or other components forming the flange 720 or flared end at the distal end 718 of the implant 700 may include a similar or comparable profile to that illustrated in FIGS. 5 and/or 6. For example, the frame 702 may include one or more cells with arms extending beyond the remainder of the frame 702 at the distal end 718 of the implant 700.

In some embodiments, the flange 720 may be generally normal to the central axis 750. Additionally or alternatively, the flange 720 may be generally centered around the line depicting the central axis 750.

FIGS. 7B-7F illustrate an example deployment of the implant 700 illustrated in FIG. 7A. The implant 700 may traverse a circulatory system 716 of a patient to arrive at a target deployment location.

As illustrated in FIG. 7B, a catheter (not illustrated) carrying the implant 700 may traverse the circulatory system 716 towards a deployment location. For example, as illustrated in FIG. 7B, it may be desired to deploy the implant 700 between the IVC and the RA. The implant 700 may traverse the circulatory system 716 in a distal direction conveyed by the arrow 751. In some embodiments, the implant 700 may be included in a delivery capsule or may be deployable via a balloon or other deployment mechanism.

As illustrated in FIG. 7C, the implant 700 may begin to pass through the IVC and into the RA as it continues along the distal direction of travel conveyed by the arrow 752.

As illustrated in FIG. 7D, upon reaching the deployment or target location, the implant 700 may be either partially or fully deployed. For example, a sheath or outer covering of the delivery capsule associated with the catheter may be moved proximally relative to the implant 700, exposing the flange 720 and/or other portions of the implant 700. The radial expansion of the implant 700 during deployment is conveyed by the arrows 753a and 753b. While illustrated as being partially within the RA and partially within the IVC, it will be appreciated that the implant 700 may be fully within the RA when it is partially or fully deployed.

As illustrated in FIG. 7E, the implant 700 may be deployed until the flange 720 is substantially or fully deployed. The continued deployment is illustrated by the arrows 754a and 754b. In some embodiments, the implant 700 may be deployed until a radial edge of the flange 720 extends beyond the circumference of the IVC. Such deployment may include full or partial deployment of the flange 720 and/or full or partial deployment of the implant 700.

In some embodiments, the stage of deployment illustrated in FIG. 7E may include the implant 700 being at least 30% deployed, at least 40% deployed, at least 50% deployed, at least 60% deployed, at least 70% deployed, at least 80% deployed, at least 90% deployed, or 100% deployed (or fully deployed). Additionally or alternatively, the stage of deployment illustrated in FIG. 7E may include the flange 720 portion of the implant 700 being at least 30% deployed, at least 40% deployed, at least 50% deployed, at least 60% deployed, at least 70% deployed, at least 80% deployed, at least 90% deployed, or 100% deployed (or fully deployed).

As illustrated in FIG. 7F, after the stage of deployment illustrated in FIG. 7E is reached, the catheter may be drawn back in the proximal direction as illustrated by the arrow 755. Doing so may facilitate the flange 720 seating against the wall of the RA to create a seal.

In some embodiments, after seating the flange 720 against the wall of the RA, the implant 700 may be deployed the remainder of the way to be fully deployed. Additionally, the implant 700 may be disengaged from the catheter and the catheter may be retrieved from the body of the patient. For example, a coupling foot of the frame 702 may be decoupled from an attachment feature of the delivery capsule. Additionally or alternatively, after the implant 700 is fully deployed, the valve may traverse the circulatory system 716 of the patient and be affixed to the implant 700 (such as by being coupled to a catheter in a valve delivery capsule, traversing the circulatory system 716 of the patient until the valve may be deployed from the valve delivery capsule and affixed to the implant 700, and the catheter and valve delivery capsule retrieved).

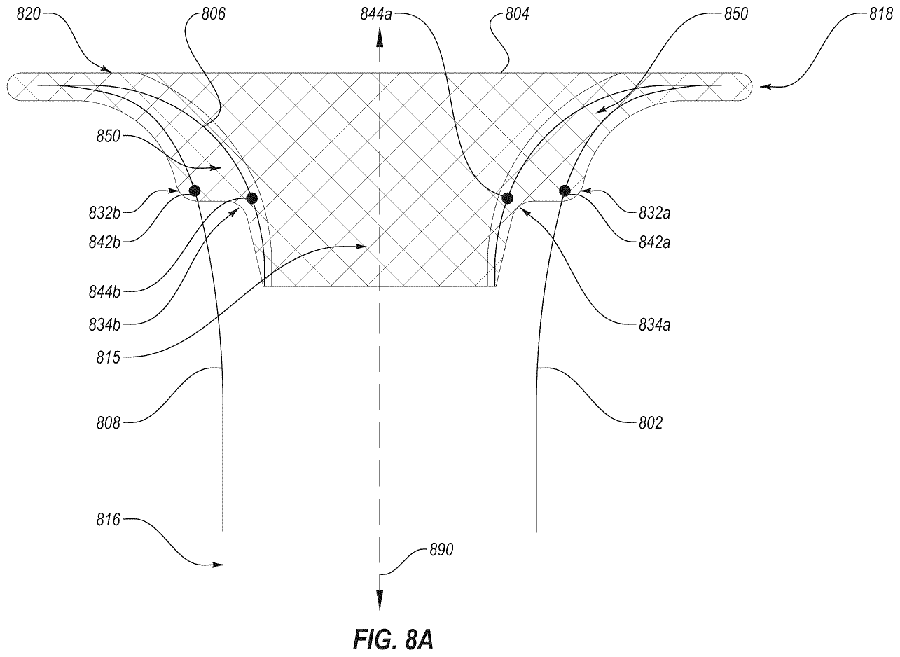

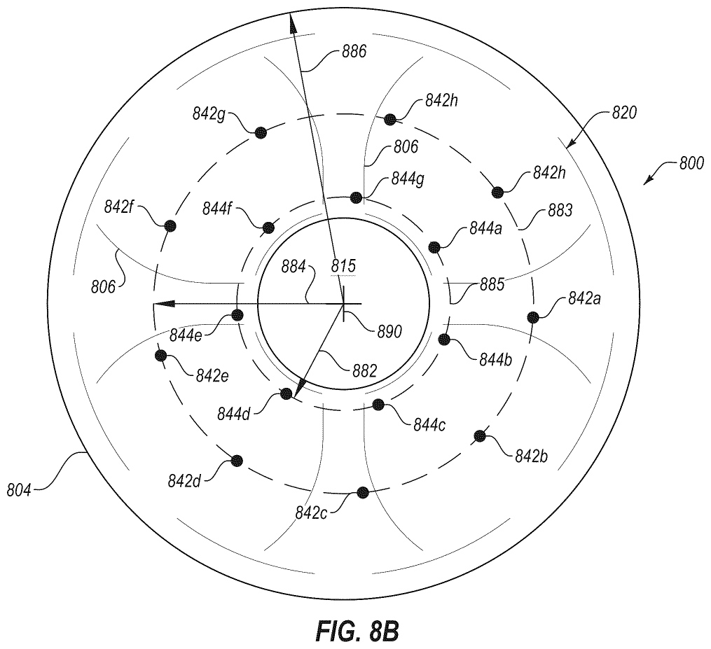

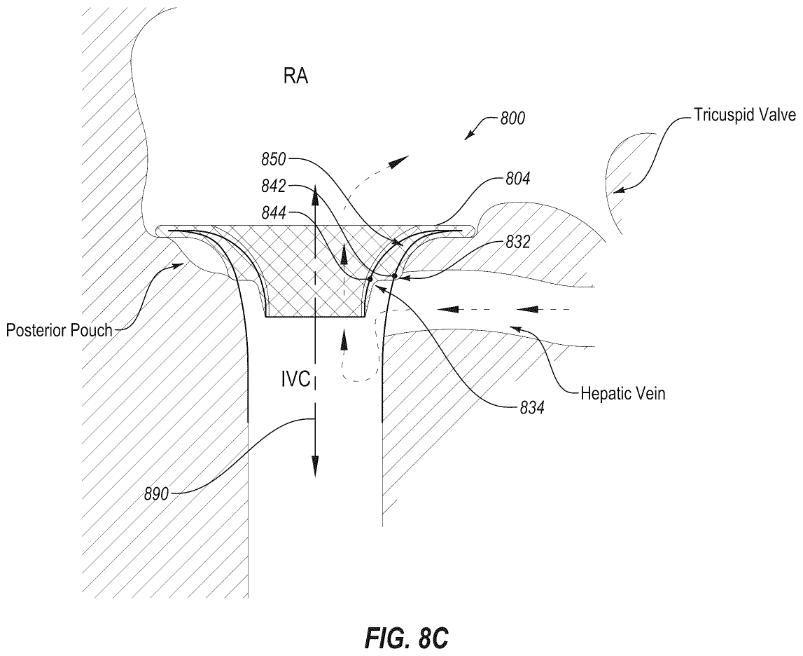

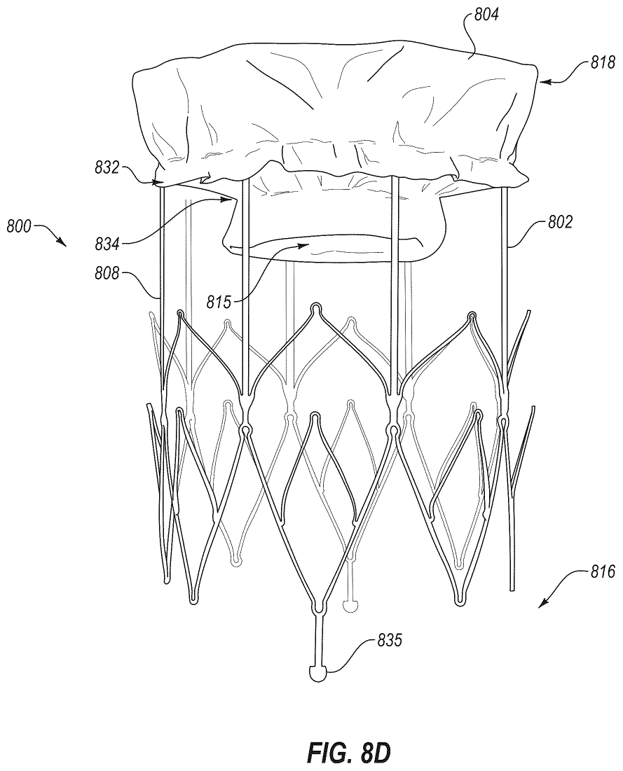

FIG. 8A illustrates a cut-away front view of an example implant 800 configured to dock and/or support one or more prosthetic valves and/or valve components with a sealing skirt 804 enclosing an annular region 850. The implant 800 includes an expandable frame 802 that is configured to expand from a compressed state into an expanded state when deployed. FIG. 8B illustrates a top-down view of the implant 800 of FIG. 8A. FIG. 8C illustrates a cut-away front view of the implant 800 of FIG. 8A when deployed within a patient. FIG. 8D illustrates a front view of the implant 800 of FIG. 8A.

The implant 800 may be similar or comparable in some regards to one or more of the implants 500 of FIGS. 5, 600 of FIG. 6, and/or 700 of FIGS. 7A-7F, among other embodiments of the present disclosure. For example, the frame 802 may be similar or comparable to the frame 502 and/or 602; the sealing skirt 804 may be similar or comparable to sealing element 504, 604, and/or 704; inner struts 806 may be similar or comparable to the inner frame 506 and/or 606; outer struts 808 may be similar or comparable to the outer frame 508 and/or 608; a valve seat 815 may be similar or comparable to the valve seat 715; proximal end 816 may be similar or comparable to the proximal ends 516 and/or 616; distal end 818 may be similar or comparable to the distal ends 518 and/or 618; and a flange 820 may be similar or comparable to the flange 720.

As illustrated in FIG. 8A, the implant 800 may include the sealing skirt that encloses the annular region 850. For example, the sealing skirt 804 may (beginning at an inner surface of the proximal end 816 of the valve seat 815) extend along and cover the inner struts 806 until the distal end 818 of the implant 800 is reached, and/or until a transition point between the inner struts 806 and the outer struts 808 is reached. This point may be at a radial edge of the flange 820 (e.g., at a flange radius 886 from a central longitudinal axis 890). From the distal end 818 of the implant 800, the sealing skirt 804 may continue along the outside struts 808 for a first span until a first elbow 832 is reached. From the first elbow 832, the sealing skirt 804 may extend back radially inwards to the inner struts 806. From the inner struts 806, the sealing skirt 804 may continue along the inner struts 806 towards the proximal end 816 of the valve seat 815. When reaching the proximal end 816 of the valve seat 815, the sealing skirt 804 may enclose an entire annular region 850.

In some embodiments, the annular region 850 may include a region about the valve seat 815 between the inner struts 806 and the outer struts 808, and between the flange 820 and a proximal end 816 of the valve seat 815. The sealing skirt 804 may fully or partially enclose the annular region 850 such that blood flow into the annular region 850 may be limited or even reduced completely. Additionally or alternatively, to the extent some blood does seep into or otherwise enter the annular region 850, it is possible the blood within the annular region may stagnate and form clots. In these and other embodiments, the sealing skirt 804 may prevent any such clots from travelling outside of the annular region 850. For example, the weaving of the material of which the sealing skirt 804 is made may leave gaps between fibers that prevent clots of a dangerous size from passing through the material of the sealing skirt 804. As another example, pores in the material of the sealing skirt and/or gaps around the stitching regions when coupling the sealing skirt 804 to the frame 802 may leave gaps that are small enough that clots of a dangerous size are prevented from passing through the sealing skirt 804.

In some embodiments, the implant 800 may include one or more radiopaque markers (such as a first set of radiopaque markers 842a-h and/or a second set of radiopaque markers 844a-g) to facilitate guidance, orientation, and otherwise facilitate a procedure of guiding the implant 800 through the body of the patient to the target location, deploying the implant 800, and/or otherwise positioning the implant 800 in the desired position. For example, the radiopaque markers may facilitate a view into an axial or radial orientation, state of deployment, or other information for the clinician when performing a procedure involving the implant 800. The radiopaque markers may be made of any radiopaque material, such as tantalum, bismuth, iodine, barium, or gold.

In some embodiments, the first series of radiopaque markers 842a-842h (referred to collectively as first radiopaque markers 842) may be positioned so as to coincide with the first elbow 832. For example, the first radiopaque markers 842 may be disposed on the outer struts 808 at or near the position of the first elbow 832, e.g., by being coined into an opening or gap in the outer struts 808. As another example, the first radiopaque markers 842 may be embedded into, stitched into, coined onto, attached via an adhesive, or otherwise be part of the sealing skirt 804 itself and be disposed at the first elbow 832. In these and other embodiments, the first radiopaque markers 842 may be disposed at periodic locations spaced about the circumference of the implant 800 at a generally consistent radial position from the central longitudinal axis 890. For example, the radiopaque markers 842 may follow a circular shape 883 with a radius 884 from the central longitudinal axis 890. While a given quantity of radiopaque markers are illustrated, it will be appreciated that any quantity are contemplated, such as three, four, six, eight, ten, twelve, fifteen, twenty, or any range bounded by any of the preceding values, such as between three and fifteen.

In some embodiments, the second series of radiopaque markers 844a-g (referred to collectively as second radiopaque markers 844) may be positioned so as to coincide with the second elbow 834. For example, the second radiopaque markers 844 may be disposed on the inner struts 806 at or near the position of the second elbow 834. Additionally or alternatively, the second set of radiopaque markers 844 may be disposed at a proximal end of the inner struts 806. In these and other embodiments, the second radiopaque markers 844 may be coined into an opening or gap in the inner struts 806. As another example, the second radiopaque markers 844 may be embedded into, stitched into, coined onto, attached via an adhesive, or may otherwise be part of or coupled to the sealing skirt 804 itself and be disposed at or near the second elbow 834. In these and other embodiments, the second radiopaque markers 844 may be disposed at periodic locations spaced about the circumference of the implant 800 at a generally consistent radial position from the central longitudinal axis 890 that is closer to the central longitudinal axis 890 than the first radiopaque markers 842. For example, the second radiopaque markers 844 may follow a circular shape 885 with a radius 882 from the central longitudinal axis 890 and the radiopaque markers 844 may be disposed at circumferential points. While a given quantity of radiopaque markers are illustrated, it will be appreciated that any quantity are contemplated, such as three, four, six, eight, ten, twelve, fifteen, twenty, or any range bounded by any of the preceding values, such as between three and fifteen.

In some embodiments, the first radiopaque markers 842 and/or the second radiopaque markers 844 may facilitate placement of the implant 800 when deploying the implant 800. For example, with reference to FIG. 8C, the implant 800 may be positioned proximate the boundary between the IVC and the RA. A clinician, using fluoroscopy or other imaging/visualization technique, may position the implant 800 such that the first radiopaque markers 842 corresponding to the first elbow 832 is positioned at or near an edge of a hepatic vein on the RA side. By doing so, the implant 800 may be positioned such that the implant 800 does not occlude the hepatic vein as the span of the sealing skirt 804 between the first elbow 832 and the second elbow 834 allows the blood to flow parallel to that span and down below the valve seat 815. Additionally or alternatively, by enclosing the annular region 850, the sealing skirt 804 may prevent the pooling or stagnation of blood in the region between the edge of the hepatic vein on the RA side and the distal end 818 of the implant 800 (e.g., the area between the flange 820 and the edge of the hepatic vein on the RA side).

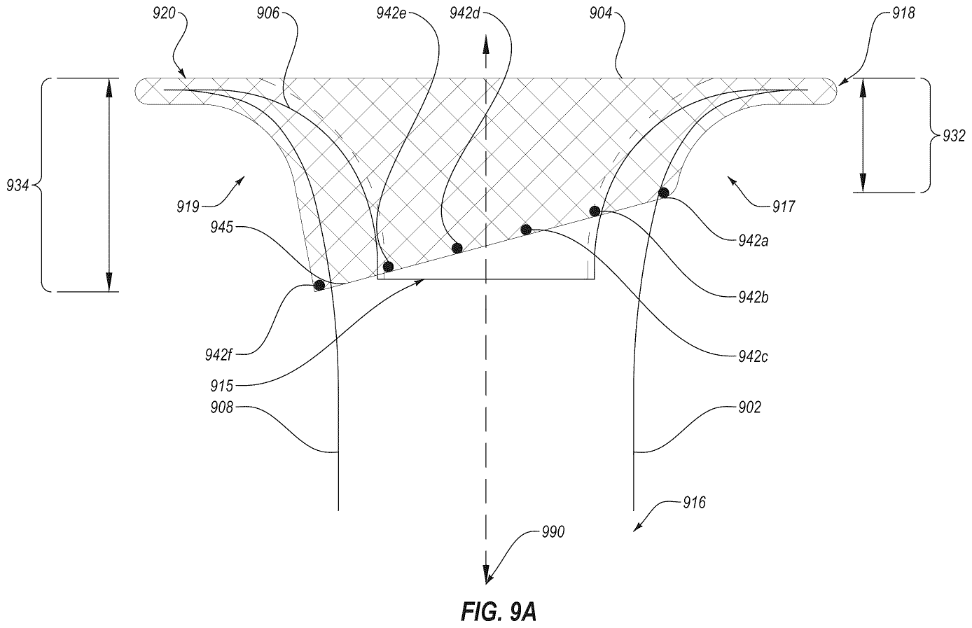

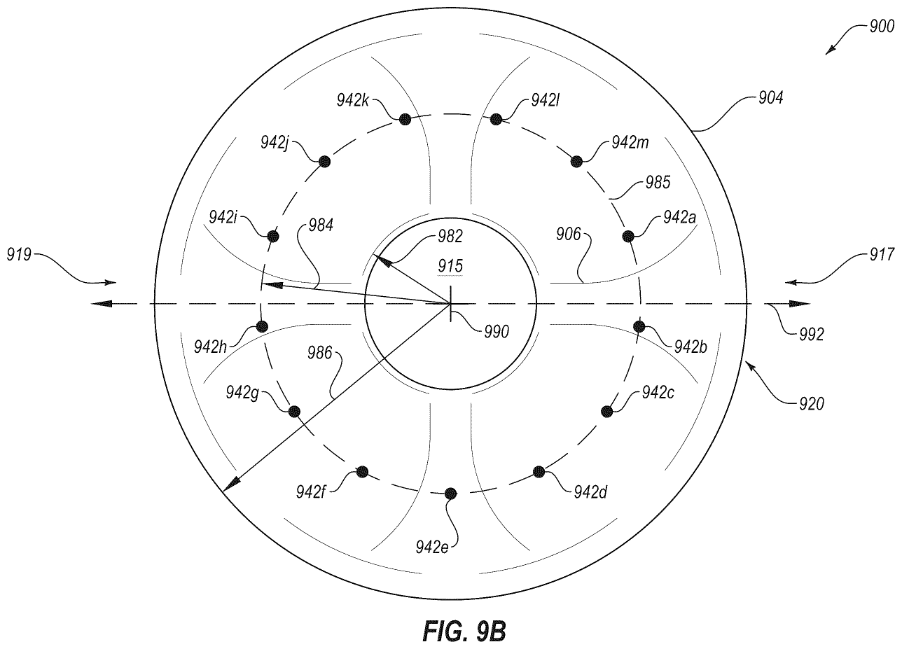

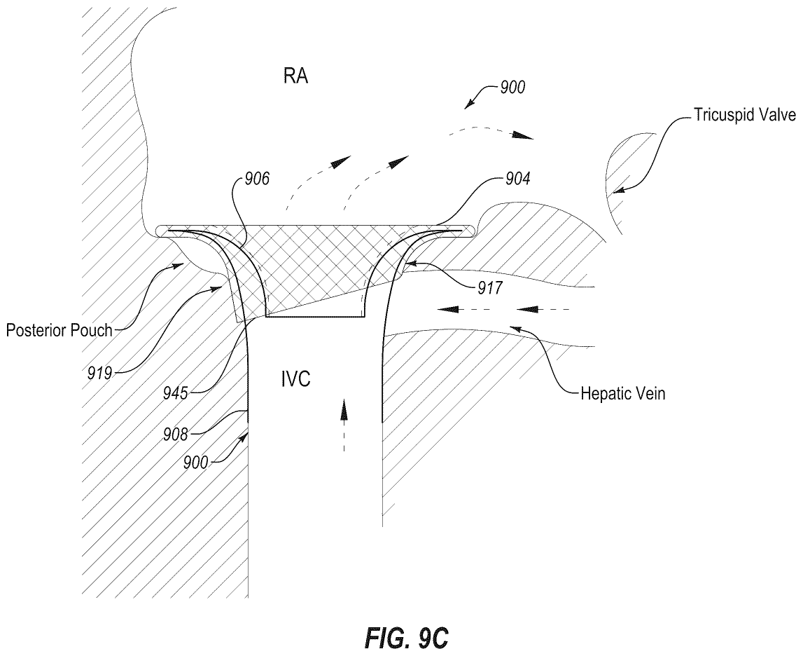

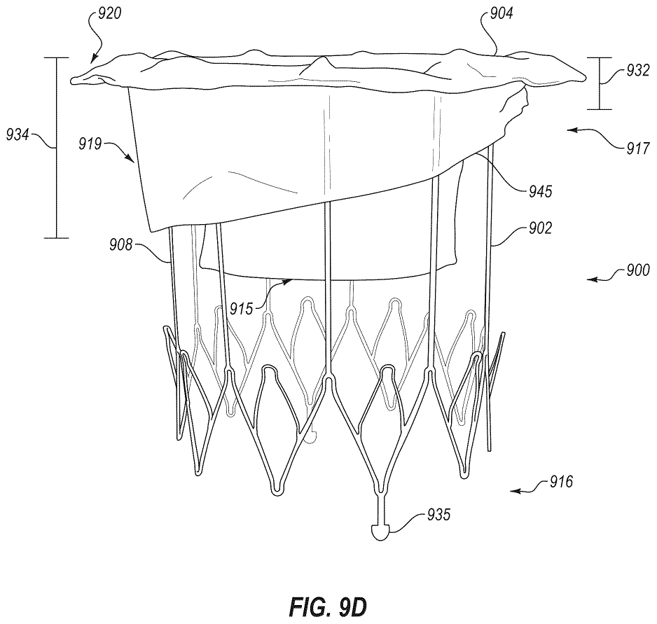

FIG. 9A illustrates a cut-away front view of an example implant 900 configured to dock and/or support one or more prosthetic valves and/or valve components with an asymmetric sealing skirt 904. The implant 900 includes an expandable frame 902 that is configured to expand from a compressed state into an expanded state when deployed. FIG. 9B illustrates a top-down view of the implant 900 of FIG. 9A. FIG. 9C illustrates a cut-away front view of the implant 900 of FIG. 9A when deployed within a patient. FIG. 9D illustrates a front view of the implant 900 of FIG. 9A.

The implant 900 may be similar or comparable in some regards to one or more of the implants 500 of FIGS. 5, 600 of FIGS. 6, 700 of FIGS. 7A-7F, and/or 800 of FIGS. 8A-8D, among other embodiments of the present disclosure. For example, the frame 902 may be similar or comparable to the frame 502, 602, and/or 802; the sealing skirt 904 may be similar or comparable to sealing element 504, 604, and/or 704 and/or the sealing skirt 804; inner struts 906 may be similar or comparable to the inner frame 506 and/or 606 and/or the inner struts 806; outer struts 808 may be similar or comparable to the outer frame 508 and/or 608 and/or the outer struts 808; a valve seat 915 may be similar or comparable to the valve seat 715 and/or the valve seat 815; proximal end 916 may be similar or comparable to the proximal ends 516, 616, and/or 816; distal end 918 may be similar or comparable to the distal ends 518, 618 and/or 818; and a flange 920 may be similar or comparable to the flange 720 and/or 820.

As illustrated in FIG. 9A, the sealing skirt 904 may be asymmetrical in form such that on a first side 917 of the implant 900, the sealing skirt 904 extends a first distance 932 from the distal end 918 of the implant along the outer struts 908 while extending a further, second distance 934 from the distal end 918 of the implant 900 along the outer struts 908 on a second side 919. In these and other embodiments, a peripheral edge 945 of the sealing skirt 904 may extend around the circumference of the implant 900, e.g., from the first side 917 to the second side 919 and back to the first side 917 (not shown). In some embodiments, at the first side 917 (e.g., for the first distance 932), the sealing skirt 904 may extend along the outer struts 908 while the outer struts 908 curve inwards from the flange 920 until approximately where the outer struts 908 extend generally parallel with the central longitudinal axis 990 when the implant 900 is fully deployed. In some embodiments, at the second side 919 (e.g., for the second distance 934), the sealing skirt 904 may extend along the outer struts 908 until approximately where the inner struts 906 stop, or stated another way, the proximal end of the valve seat 915.

In some embodiments, the first distance 932 may be characterized relative to the second distance 934. For example, the first distance 932 may be 75%, 60%, 50% (e.g. half), 40%, 33% (e.g., one third), 30%, 25% (e.g., one fourth), 20% (e.g., one fifth), 15%, 10% (e.g., one tenth), or smaller, of the second distance 934. In some embodiments, the first distance 932 may be characterized as a set distance. For example, the first distance may be 3 millimeters (mm) or less, 5 mm or less, 7 mm or less, 10 mm or less, 12 mm or less, 15 mm or less, 20 mm or less, 25 mm or less, 30 mm or less, or any range using the disclosed distances as the end points, such as between 15 mm and 5 mm.

As illustrated in FIG. 9A, the asymmetry of the sealing skirt 904 results in the first distance 932 being shorter at the first side 917 while the second distance 934 is longer at the second side 919. By providing the asymmetrical sealing skirt 904, the implant 900 may be oriented such that the shorter first side 917 may be positioned near the hepatic veins so as to reduce the risk of occlusion of the hepatic veins while the longer second side 919 may be positioned near the posterior pouch to create a more stable seal against the anatomy of the patient. For example, the longer second side 919 may be able to span a cavity created by the posterior pouch to create a stable seal despite the variations in size and contour of the cavity formed by the posterior pouch. In these and other embodiments, the flange 920 of the implant 900 may be positioned within the RA (e.g., may be located downstream from the IVC and the posterior pouch) such that the flange 920 may press against the anatomy of the patient within the RA and the longer second side 919 of the sealing skirt 904 may span from within the RA to beyond the cavity formed by the posterior pouch. By doing so, the implant 900 may create a stable seal to prevent backflow into the IVC while also helping to reduce or prevent stagnation of blood proximate an implant within the cavity formed by the posterior pouch.

FIG. 9C illustrates the implant 900 deployed at the juncture between the IVC and the RA. As illustrated in FIG. 9C, the implant 900 may be oriented with the first side 917 positioned near the hepatic veins and the second side 919 positioned against the posterior pouch.

In some embodiments, the implant 900 may include radiopaque markers 942 (such as the radiopaque markers 942a-942m). The radiopaque markers 942 may facilitate guidance, orientation, and otherwise facilitate a procedure of guiding the implant 900 through the body of the patient to the target location, deploying the implant 900, and/or otherwise positioning the implant 900 in the desired position and/or orientation. For example, the radiopaque markers 942 may facilitate a view into an axial or radial orientation, state of deployment, or other information for the clinician when performing a procedure involving the implant 900.

In some embodiments, the radiopaque markers 942 may be disposed along the peripheral edge 945 of the sealing skirt 904. For example, the radiopaque markers 942 may be disposed on the outer struts 908 at or near the position of the peripheral edge 945, e.g., by being coined into an opening or gap in the outer struts 908. As another example, the radiopaque markers 942 may be embedded into, stitched into, coined onto, attached via an adhesive, or otherwise be part of the sealing skirt 904 itself and be disposed along the peripheral edge 945. In these and other embodiments, the radiopaque markers 942 may be disposed at periodic locations spaced about the circumference of the implant 900 at a generally consistent radial position from the central longitudinal axis 990 at circumferential points. For example, as illustrated in FIG. 9B, the valve seat 915 and/or a proximal end of the inner struts 906 may be disposed at a first radius 982 (e.g., a valve radius), the radiopaque markers 942 may be disposed at a second radius 984 (e.g., a marker radius), and the edge of the flange 920 may be at a third radius 986 (e.g., a flange radius).

In some embodiments, the radiopaque markers 942 may be asymmetrically spaced about the circumference of the implant 900 at circumferential points. For example, the radiopaque markers 942 may be closer together at the first side 917 and further apart at the second side 919. By doing so, a clinician or other person performing a procedure may be able to visually observe the orientation of the implant 900 from both a side view (such as that illustrated in FIG. 9A) or a top or bottom view (such as the top view illustrated in FIG. 9B). For example, from the side view, the clinician may be able to observe the descending line formed by the radiopaque markers 942 as they follow the peripheral edge 945 of the sealing skirt 904 from the first side 917 to the second side 919. As another example, from the top view, the clinician may be able to observe the radiopaque markers 942 more densely spaced at the first side 917 and more loosely spaced at the second side 919.

While a given quantity of radiopaque markers 942 are illustrated, it will be appreciated that any quantity of radiopaque markers 942 is contemplated, such as three, four, six, eight, ten, twelve, fifteen, twenty, or any range bounded by any of the preceding values, such as between three and fifteen radiopaque markers 942.

In some embodiments, the implant 900, including the sealing skirt 904, may be symmetrical about a first plane extending through a line 992 spanning from the first side 917 to the second side 919 and the central longitudinal axis 990. In these and other embodiments, the implant 900, excluding the sealing skirt 904, may also be symmetrical about a second plane passing through the central longitudinal axis 990 and orthogonal to the line 992.

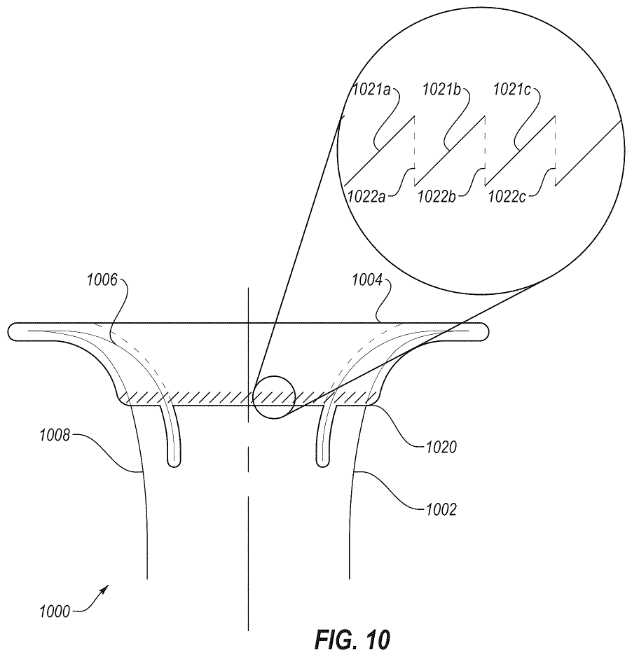

FIG. 10 illustrates a cut-away front view of an example implant 1000 configured to dock and/or support one or more prosthetic valves and/or valve components with improved stitches 1020 for a sealing skirt 1004. The implant 1000 includes an expandable frame 1002 that is configured to expand from a compressed state into an expanded state when deployed.

The implant 1000 may be similar or comparable in some regards to one or more of the implants 500 of FIGS. 5, 600 of FIGS. 6, 700 of FIGS. 7A-7F, 800 of FIGS. 8A-8D, and/or 900 of FIGS. 9A-9D, among other embodiments of the present disclosure. For example, the frame 1002 may be similar or comparable to the frame 502, 602, 802, and/or 902; the sealing skirt 1004 may be similar or comparable to sealing element 504, 604, and/or 704 and/or the sealing skirt 804 and/or 904; inner struts 1006 may be similar or comparable to the inner frame 506 and/or 606 and/or the inner struts 806 and/or 906; outer struts 1008 may be similar or comparable to the outer frame 508 and/or 608 and/or the outer struts 808 and/or 908.

As illustrated in FIG. 10, the stitches 1020 may be used to attach the sealing skirt 1004 to the frame 1002. For example, the sealing skirt 1004 may overlay part or all of the frame 1002, such as a flange at one end of the implant 1000. In some embodiments, the stitches 1020 may be disposed along a peripheral edge of the sealing skirt 1004 to affix the sealing skirt to the frame 1002.

In some embodiments, the stitches 1020 may include a series of alternating angled stitches 1021 followed by longitudinal stitches 1022. For example, FIG. 10 illustrates a first angled stitch 1021a followed by a first longitudinal stitch 1022a followed by a second angled stitch 1021b followed by a second longitudinal stitch 1022b followed by a third angled stitch 1021c followed by a third longitudinal stitch 1022c. In some embodiments, the stitches 1020 may be disposed along a peripheral edge of the sealing skirt 1004.

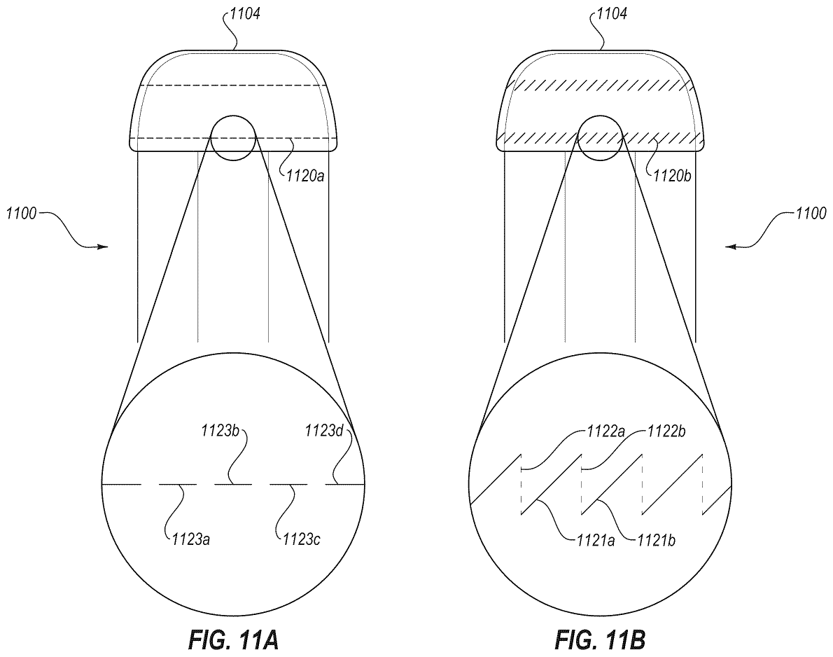

By providing stitches 1020 that are at least partially oriented in the longitudinal direction of the implant 1000, certain forces and loading difficulties may be mitigated or avoided. For example, when loading the implant 1000 into a delivery capsule (not shown), the frame 1002 is compressed down. Laterally oriented stitches (such as those illustrated in FIG. 11A) may snag or catch when being compressed into the delivery capsule or when being released out of the delivery capsule when deployed. This may create undue strain on the implant 1000 and/or the sealing skirt 1004 as the stitches 1020 catch on the delivery capsule. For example, if the stitches snag on the delivery capsule, the implant 1000 may bend, fold, or otherwise be deformed in a manner that may result in material failure of the implant 1000. As another example, if the stitches snag on the delivery capsule, the implant 1000 may expand in an incomplete or improper manner when being deployed. As a further example, if the stitches snag on the delivery capsule, the sealing skirt 1004 may be disengaged from the implant 1000 or a hole or gap may form in the sealing skirt 1004, which may result in blood leaking past the implant 1000 without passing through a valve (not illustrated) deployed into the implant 1000. By providing the angled stitches 1021 and/or longitudinal stitches 1022, the stitches 1020 may more readily slide past the delivery capsule when loading the implant 1000 into the delivery capsule. Additionally, the stitches 1020 may more readily slide past the delivery capsule when the implant 1000 is deployed as compared to laterally oriented stitches.

While illustrated with the angled stitches 1021 being oriented on an outside of the implant 1000 with the longitudinal stitches 1022 located on an inside of the implant 1000, it will be appreciated that the inverse is also contemplated in which the longitudinal stitches 1022 may be on the outside of the implant 1000 and the angled stitches 1021 may be located on the inside of the implant 1000.

FIG. 11A illustrate a front view of another example implant 1100 configured to dock and/or support one or more prosthetic valves and/or valve components with lateral stitches 1120a for a sealing skirt 1104. FIG. 11B illustrate a front view of the implant of FIG. 11A with improved stitches 1120b for the sealing skirt 1104.

The implant 1100 may be similar or comparable in some regards to one or more of the implants 500 of FIG. 5, 600 of FIG. 6, 700 of FIGS. 7A-7F, 800 of FIGS. 8A-8D, 900 of FIGS. 9A-9D, and/or 1000 of FIG. 10, among other embodiments of the present disclosure. For example, the sealing skirt 1004 may be similar or comparable to sealing element 504, 604, and/or 704 and/or the sealing skirt 804, 904, and/or 1004; additionally or alternatively, the stitches 1120b may be similar or comparable to the stitches 1020.

As illustrated in FIG. 11A, the lateral stitches 1120a may be an example of stitches that may be more likely to snag on a delivery capsule when loading the implant 1100 into the delivery capsule or when deploying the implant 1100 out of the delivery capsule.

As illustrated in FIG. 11B, the stitches 1120b include another example of angled stitches 1121 and longitudinal stitches 1122 that may facilitate loading and/or deploying the implant 1100. For example, the stitches 1120b may include alternating angled stitches 1121 and longitudinal stitches 1122.

In some embodiments, the stitches 1120 may include a single row of stitches. Additionally or alternatively, the stitches 1120 may include multiple rows of stitches 1120. For example, the stitches 1120 may include two, three, four, or more rows of stitches.

In some embodiments, the improved stitches may be applicable to different types of implants. For example, as illustrated in FIG. 10, the improved stitches 1020 may be used in conjunction with the implant 1000 with the flare at one end of the implant 1000. As another example, as illustrated in FIG. 11B, the improved stitches 1120 may be used in conjunction with the implant 1100 that tapers radially inward at one end of the implant 1100.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implant comprising: a frame comprising: outer struts to interface with a patient; and inner struts that form a valve seat within a central cavity of the frame; and a sealing skirt overlaying the frame from the valve seat to the outer struts at a distal end of the frame, extending along a span of the outer struts, and then extending back towards the inner struts to enclose an annular region about the valve seat.

Example 2. Any of the foregoing examples, such as example 1, wherein the sealing skirt further extends along a portion of the inner struts after extending back towards the inner struts.

Example 3. Any of the foregoing examples, such as example 2, wherein the sealing skirt creates a first elbow where the sealing skirt extends back towards the valve seat.

Example 4. Any of the foregoing examples, such as example 3, further comprising a first radiopaque marker disposed in the first elbow.

Example 5. Any of the foregoing examples, such as example 4, further comprising a first series of radiopaque markers disposed at a first series of circumferential points about the frame, the first series of radiopaque markers including the first radiopaque marker, and the first series of circumferential points being radially equidistant from a central longitudinal axis of the frame.

Example 6. Any of the foregoing examples, such as any of examples 3-5, wherein the sealing skirt creates a second elbow where the sealing skirt reaches the inner struts.

Example 7. Any of the foregoing examples, such as example 6, further comprising a second radiopaque marker disposed in the second elbow.

Example 8. Any of the foregoing examples, such as example 7, further comprising a second series of radiopaque markers disposed at a second series of circumferential points about the frame, the second series of radiopaque markers including the second radiopaque marker, and the second series of circumferential points being radially equidistant from a central longitudinal axis of the frame.

Example 9. Any of the foregoing examples, such as any of examples 3-8, wherein the first elbow is closer to the distal end of the frame than a proximal end of the valve seat.

Example 10. Any of the foregoing examples, such as any of examples 1-9, wherein the frame forms a flange extending radially outward at the distal end of the frame, and wherein the sealing skirt overlays the flange.

Example 11. Any of the foregoing examples, such as any of examples 1-10, wherein the sealing skirt prevents blood flow into the annular region.

Example 12. A method comprising: extending a delivery capsule with an implant loaded thereon through a patient in a first direction to a target location; at least partially deploying the implant at the target location, the implant including a frame that includes outer struts and inner struts that form a valve seat within a central cavity of the frame, the implant also including a sealing skirt overlaying the frame from the valve seat to the outer struts at a distal end of the frame, the sealing skirt extending along a span of the outer struts, and then the sealing skirt extending back towards the inner struts to enclose an annular region about the valve seat; and disengaging the implant from the delivery capsule.

Example 13. Any of the foregoing examples, such as example 12, further comprising pulling the implant back in a second direction opposite of the first direction to seat the distal end of the frame against anatomy of the patient to create a seal between the implant and the anatomy.

Example 14. Any of the foregoing examples, such as example 13, further comprising, after seating the distal end of the frame against the anatomy, completing any remaining deployment of the implant.

Example 15. Any of the foregoing examples, such as example 14, wherein completing any remaining deployment of the implant comprises releasing a foot of the frame from the delivery capsule and retracting the delivery capsule back through the patient.

Example 16. Any of the foregoing examples, such as any of examples 12-14, further comprising installing a valve within the frame at the valve seat.

Example 17. Any of the foregoing examples, such as example 16, wherein installing the valve comprises: extending a valve delivery capsule with the valve loaded thereon through the patient; deploying the valve within the implant; and fixing the valve to the implant in the valve seat.

Example 18. Any of the foregoing examples, such as any of examples 12-17, wherein the target location is within a right atrium (RA), an inferior vena cava (IVC), or both, of the patient.

Example 19. Any of the foregoing examples, such as example 12-18, wherein the target location positions the valve seat at a boundary between a RA and an IVC of the patient.

Example 20. An implant comprising: a frame comprising: outer struts to interface with a patient; and inner struts that form a valve seat within a central cavity of the frame; and a sealing skirt overlaying the frame from the valve seat to the outer struts at a distal end of the frame, the sealing skirt including a first side that extends a first distance from the distal end of the frame along the outer struts and a second side that extends a second distance from the distal end of the frame along the outer struts, the second distance longer than the first distance.

Example 21. Any of the foregoing examples, such as example 20, wherein the first distance is one half the second distance or less.

Example 22. Any of the foregoing examples, such as any of examples 20-21, wherein the first distance is one third the second distance or less.

Example 23. Any of the foregoing examples, such as any of examples 20-22, wherein the first distance is ten millimeters (mm) or less.

Example 24. Any of the foregoing examples, such as any of examples 20-23, further comprising a first series of radiopaque markers disposed along a peripheral edge of the sealing skirt.

Example 25. Any of the foregoing examples, such as example 24, wherein the peripheral edge of the sealing skirt traverses along the outer struts and is at the first distance from the distal end of the frame on the first side and progresses to the second distance from the distal end of the frame on the second side.

Example 26. Any of the foregoing examples, such as any of examples 24-25, wherein the first series of radiopaque markers form a circular shape with a radius smaller than a flange radius of a distal end of the frame, and with the radius larger than a valve radius of the valve seat.

Example 27. Any of the foregoing examples, such as any of examples 20-26, wherein the second distance is a same distance as a proximal end of the valve seat from the distal end of the frame.

Example 28. Any of the foregoing examples, such as any of examples 20-27, further comprising a valve deployed in the valve seat.

Example 29. Any of the foregoing examples, such as any of examples 20-28, wherein the frame forms a flange extending radially outward at the distal end of the frame.

Example 30. Any of the foregoing examples, such as any of examples 20-29, wherein the second distance is a same distance as a distance the inner struts extend from the distal end of the frame.

Example 31. Any of the foregoing examples, such as any of examples 20-30, wherein the implant is symmetrical about a plane extending from the first side to the second side and passing through a central longitudinal axis passing longitudinally through a center of the implant.

Example 32. Any of the foregoing examples, such as any of examples 20-31, wherein the sealing skirt is symmetrical about a longitudinal plane extending from the first side to the second side and passing through a longitudinal axis passing longitudinally through a center of the implant.

Example 33. A method comprising: extending a delivery capsule with an implant loaded thereon through a patient in a first direction to a target location; at least partially deploying the implant at the target location, the implant including an frame that includes outer struts and inner struts that form a valve seat within a central cavity of the frame, the implant also including a sealing skirt overlaying the frame from the valve seat to the outer struts at a distal end of the frame, the sealing skirt including a first side that extends a first distance from the distal end of the frame along the outer struts and a second side that extends a second distance from the distal end of the frame along the outer struts, the second distance longer than the first distance; orienting the implant in the target location such that the first side is proximate a hepatic vein; and disengaging the implant from the delivery capsule.

Example 34. Any of the foregoing examples, such as example 33, further comprising pulling the implant back in a second direction opposite of the first direction to seat the distal end of the frame against anatomy of the patient to create a seal between the implant and the anatomy.

Example 35. Any of the foregoing examples, such as example 34, after seating the distal end of the frame against the anatomy, completing any remaining deployment of the implant.

Example 36. Any of the foregoing examples, such as example 35, wherein completing any remaining deployment of the implant comprises releasing a foot of the frame from the delivery capsule and retracting the delivery capsule back through the patient.

Example 37. Any of the foregoing examples, such as any of examples 33-36, further comprising installing a valve within the frame at the valve seat.

Example 38. Any of the foregoing examples, such as example 37, wherein installing the valve comprises: extending a valve delivery capsule with the valve loaded thereon through the patient; deploying the valve within the implant; and fixing the valve to the implant in the valve seat.

Example 39. Any of the foregoing examples, such as any of examples 33-38, wherein the target location is within a right atrium (RA), an inferior vena cava (IVC), or both, of the patient.

Example 40. Any of the foregoing examples, such as any of examples 33-39, wherein, when the implant is fully deployed, the valve seat is positioned at a boundary between a RA and an IVC of the patient.

Example 41. Any of the foregoing examples, such as any of examples 33-40, wherein orienting the implant in the target location such that the first side is proximate a hepatic vein includes: observing image capturing radiopaque markers disposed along a peripheral edge of the sealing skirt; and rotating the implant such that the first side of the implant is proximate the hepatic vein.

Example 42. Any of the foregoing examples, such as example 41, wherein the image includes a fluoroscopy image.

Example 43. Any of the foregoing examples, such as any of examples 33-42, wherein orienting the implant in the target location such that the first side is proximate a hepatic vein includes positioning the implant in the target location such that the second side is proximate a posterior pouch of the patient.

Example 44. An implant comprising: a frame comprising: outer struts; and inner struts that form a valve seat within a central cavity of the frame; and a sealing skirt overlaying the frame from the valve seat to the outer struts at a distal end of the frame, the sealing skirt coupled to the frame using stitches that are at least partially oriented in a longitudinal direction of the frame.

Example 45. Any of the foregoing examples, such as example 44, wherein the stitches include a row of alternating stitches that include a longitudinal stitch followed by an angled stitch.

Example 46. Any of the foregoing examples, such as any of examples 44-45, wherein the frame forms a flange extending radially outward at the distal end of the frame, and wherein the sealing skirt overlays the flange.

Example 47. Any of the foregoing examples, such as any of examples 44-46, wherein the frame tapers radially inward at the distal end of the frame.

Example 48. Any of the foregoing examples, such as any of examples 44-47, wherein the stitches include a single row of stitches.

Example 49. Any of the foregoing examples, such as any of examples 44-47, wherein the stitches include at least two rows of stitches.

Example 50. Any of the foregoing examples, such as any of examples 44-49, wherein the stitches are disposed at a peripheral edge of the sealing skirt.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims. We therefore claim all that comes within the scope and spirit of these claims.

1. An implant comprising:
a frame comprising:
outer struts to interface with a patient; and
inner struts that form a valve seat within a central cavity of the frame; and
a sealing skirt overlaying the frame from the valve seat to the outer struts at a distal end of the frame, extending along a span of the outer struts, and then extending back towards the inner struts to enclose an annular region about the valve seat.

2. The implant of claim 1 wherein the sealing skirt further extends along a portion of the inner struts after extending back toward the inner struts and the sealing skirt creates a first elbow.

3. The implant of claim 2, further comprising a first radiopaque marker disposed in the first elbow.

4. The implant of claim 1, further comprising a first series of radiopaque markers disposed at a first series of circumferential points about the frame, the first series of radiopaque markers including the first radiopaque marker, and the first series of circumferential points being radially equidistant from a central longitudinal axis of the frame.

5. The implant of claim 2, wherein the sealing skirt creates a second elbow where the sealing skirt reaches the inner struts.

6. The implant of claim 5, further comprising a second radiopaque marker disposed in the second elbow.

7. A method comprising:
extending a delivery capsule with an implant loaded thereon through a patient in a first direction to a target location;
at least partially deploying the implant at the target location, the implant including a frame that includes outer struts and inner struts that form a valve seat within a central cavity of the frame, the implant also including a sealing skirt overlaying the frame from the valve seat to the outer struts at a distal end of the frame, the sealing skirt extending along a span of the outer struts, and then the sealing skirt extending back towards the inner struts to enclose an annular region about the valve seat; and
disengaging the implant from the delivery capsule.

8. An implant comprising:
a frame comprising:
outer struts to interface with a patient; and
inner struts that form a valve seat within a central cavity of the frame; and
a sealing skirt overlaying the frame from the valve seat to the outer struts at a distal end of the frame, the sealing skirt including a first side that extends a first distance from the distal end of the frame along the outer struts and a second side that extends a second distance from the distal end of the frame along the outer struts, the second distance longer than the first distance.

9. The implant of claim 8 wherein the first distance is one third the second distance or less.

10. The implant of claim 8, wherein the first distance is ten millimeters (mm) or less.

11. The implant of claim 8, further comprising a first series of radiopaque markers disposed along a peripheral edge of the sealing skirt.

12. The implant of claim 11, wherein the peripheral edge of the sealing skirt traverses along the outer struts and is at the first distance from the distal end of the frame on the first side and progresses to the second distance from the distal end of the frame on the second side.

13. The implant of claim 11, wherein the first series of radiopaque markers form a circular shape with a radius smaller than a flange radius of a distal end of the frame, and with the radius larger than a valve radius of the valve seat.

14. The implant of claim 8, further comprising a valve deployed in the valve seat.

15. The implant of claim 8, wherein the frame forms a flange extending radially outward at the distal end of the frame.

16. An implant comprising:
a frame comprising:
outer struts; and
inner struts that form a valve seat within a central cavity of the frame; and
a sealing skirt overlaying the frame from the valve seat to the outer struts at a distal end of the frame, the sealing skirt coupled to the frame using stitches that are at least partially oriented in a longitudinal direction of the frame.

17. The implant of claim 16, wherein the stitches include a row of alternating stitches that include a longitudinal stitch followed by an angled stitch.

18. The implant of claim 16, wherein the frame forms a flange extending radially outward at the distal end of the frame, and wherein the sealing skirt overlays the flange.

19. The implant of claim 16, wherein the frame tapers radially inward at the distal end of the frame.

20. The implant of claim 16, wherein the stitches include at least two rows of stitches.
